(19) United States
(12) Patent Application Publication
(10) Pub. No.: US 2023/0088841 A1
(43) Pub. Date: Mar. 23, 2023

(54) Genome-Wide Detection of DNA Repeats Expanded in Disease

(71) Applicant: The Hospital for Sick Children, Toronto (CA)

(72) Inventors: Stephen Scherer, Toronto (CA); Ryan Yuen, Toronto (CA)

(21) Appl. No.: 17/787,035

(22) PCT Filed: Dec. 18, 2020
(86) PCT No.: PCT/CA2020/051762

§ 371 Date: Jun. 17, 2022

Related U.S. Application Data
(60) Provisional application No. 62/951,671, filed on Dec. 20, 2019.

(51) Int. Cl. C12Q 1/6883; G16B 30/00; G16B 20/00

(52) U.S. Cl. CPC C12Q 1/6883; G16B 30/00; G16B 20/00; C12Q 1/6869

(57) ABSTRACT

A method of detecting tandem repeat expansions associated with a disease is provided. The method includes the steps of: detecting tandem repeat sequences comprising a repeated motif sequence in nucleic acid samples from individuals within a population of interest, simulating the length distribution of the tandem repeat sequences in the population of interest to a normal distribution if no tandem repeat sequences are detected, and detecting one or more outlier tandem repeat sequences in the tandem repeat sequences detected, wherein an outlier tandem repeat sequence has a length that is greater than that in 90% of the tandem repeat sequences detected in the population interest and occur at a frequency of less than 1% of the tandem repeat sequences detected in a control population. The method is useful for the diagnosis of disease and subsequent treatment of a diagnosed individual.

### FIELD OF THE INVENTION

The present invention generally relates to genomic characteristics that correlate with disease, and in particular, to methods of detecting tandem repeats indicative of disease.

### BACKGROUND OF THE INVENTION

Technical advances in genome analysis have improved clinical diagnosis and gene discovery for human diseases that have a significant genetic component. However, for many complex disorders, including autism spectrum disorder (ASD), the causal genetic variants thus far identified generally confer less risk than expected from empirical estimates of heritability.

ASD refers to a group of neurodevelopmental disorders that are characterized by atypical social function, communication deficits, restricted interests, and repetitive behaviors. Genetic factors contribute to the etiology of ASD; twin studies estimate heritability in the 50-90% range, and recurrence in families is ˜20%. Individuals with ASD can have additional medical complications such as intellectual disability or epilepsy, and ASD itself features in many medical genetic conditions, the prototypical example being fragile X syndrome.

Genomic analyses with microarrays, exome sequencing, and genome sequencing have shown that individuals with ASD have a two- to three-fold increase in the number of rare copy number variations (CNVs) and de novo loss-of-function (nonsense, frameshift and splice site) variants, compared to their unaffected siblings. More complex structural DNA variations are also involved in ASD. With all of these studies combined, more than 100 genes and loci are known to be associated with increased likelihood of ASD. However, collectively, these genetic factors can only explain the etiology of ˜20% of ASD cases. Genome sequencing is the current state-of-the-art technology for variant detection, but even its application leaves the majority of ASD cases ‘genetically unsolved’. This “missing heritability” could, in part, be attributed to the inaccessibility of repetitive regions in DNA due to technical limitations of generating and/or resolving short-read next-generation sequence data.

Tandem repetitive DNA makes up ˜6% of the human genome. Expansions of gene-specific repeats have been linked to >40 neurodegenerative and neuromuscular genetic diseases. Alternative repeat motifs are found in some of these disorders, which complicates identification. A given tandem repeat-related gene can contribute to a variety of clinically distinct conditions. For example, the unstable CGG tract of FMR1 has been linked to intellectual delay in fragile X syndrome, fragile X premature ovarian insufficiency, fragile X associated ataxia, endocrine, autoimmune, metabolic disease, and ASD.

It would be desirable to develop an improved method for detecting and/or analyzing tandem repeat expansions in a genome.

### SUMMARY OF THE INVENTION

A comprehensive strategy for genome-wide discovery of repeat sequence expansion has been developed to identify loci as candidates for involvement in disease such as ASD.

Accordingly, in one aspect, a method of detecting one or more outlier tandem repeat sequences associated with a disease in a population of interest is provided. The method includes the following steps:

i) detecting tandem repeat sequences comprising a repeated motif sequence in nucleic acid samples from individuals within a population of interest which have been sequenced using a sequencing platform, wherein the tandem repeat sequences have a length that permits a tandem repeat calling algorithm to detect and/or genotype the tandem repeat sequences;

ii) simulating the length distribution of the tandem repeat sequences in the population of interest to a normal distribution if no tandem repeat sequences are detected in step i); and

iii) detecting one or more outlier tandem repeat sequences, wherein an outlier tandem repeat sequence has a length that is greater than that in 90% of the tandem repeat sequences detected in the population interest and occur at a frequency of less than 1% of the tandem repeat sequences detected in control population.

In another aspect, a method of diagnosing and treating an individual with a disease, such as ASD, is provided. The method comprises:

i) detecting in a nucleic acid sample from the individual the presence of one or more target outlier tandem repeat sequences determined to be prominent for that disease using nucleic acid probes that specifically bind thereto or bind to region adjacent thereto;

ii) determining that the individual has the disease when the presence of the target outlier tandem repeat sequence or sequences is present in the nucleic acid sample; and

iii) optionally, treating the individual using an appropriate therapy for the disease.

These and other aspects of the invention are described by reference to the detailed description and figures.

### BRIEF DESCRIPTION OF THE FIGURES

FIG. 1. Schematic workflow of the tandem repeat detection and analyses. ¹Tandem repeats here are defined as 2-20 bp repeat motifs that span at least 150 bp. ²Rare expansions here are defined as the tandem repeat expansions that are outliers and occur in <0.1% of individuals from the 1000 Genomes Project. Note that ExpansionHunter Denovo only approximates the size and location of a given tandem repeat; thus, we use the term “region” to refer to a genomic segment detected in this way, and reserve “location” or “locus” for sites that have been more precisely mapped.

FIG. 2. Genome analysis of tandem repeats. a, Circos plot showing the genomic distributions (1^{st} layer) of 31,793 regions with tandem repeats (2^{nd} layer), known simple sequence repeat regions (3^{rd} layer), sequence conservation (4^{th} layer), GC content (5^{th} layer), and known fragile sites (6^{th} layer). b, Nucleotide composition of the tandem repeats detected. c, Distribution of repeat unit (motif) sizes for the tandem repeats detected. d, Proportion of genic features overlapped by the tandem repeats detected. The proportion is derived from the size of tandem repeats over the total size of each genic feature. Dashed line indicates genome-average level. e, Correlation analysis between tandem repeats and different genomic features in a. By binning the genome into 1 kb windows, we tested the correlation/enrichment of different genomic features and the tandem repeats by regressing a genomic feature on the number of tandem repeats found per window. The odds ratios were derived from the logistic regression coefficients of the genomic features. Red bars represent tandem repeats detected (N=31,793 tandem repeat loci), while blue bars represent known simple sequence repeats (N=1,031,708 known short tandem repeats). Error bars indicate 95% confidence intervals. f, Validation of variable size in a tandem repeat detected. Schematic diagram (top) shows the design of a Southern blotting experiment in the targeted repeat in LINGO3, which overlaps with the location of fragile site FRA19B. Two families with different repeat sizes (3-0109 and 3-0533) are shown. In family 3-0533, the allele of size ˜125 CGG repeats in the child appears to be a contraction of the father's expanded allele, which displays multiple bands varying in repeat size (˜350, ˜450, and ˜525 CGG repeats).

FIG. 3. Functional analysis of rare (<0.1% frequency in 1000G) tandem repeat expansions. a, Burden comparison of all rare expansions, intergenic rare expansions, and genic rare expansions. Odds ratio is for ASD-affected individuals (N=1,812) compared with their unaffected siblings (N=1,485). The trend for genic expansions is preserved regardless of the frequency threshold used to define a tandem repeat expansion as rare in population controls. b, Repeat size distribution in probands, their parents, and their unaffected siblings, where the probands have rare tandem repeat expansions (N=10 families). The diagram on the left shows a zoomed-in view of the repeat-size distribution between the 99th and 100th percentile. The minima and maxima indicate 3× inter-quartile range-deviated tandem repeat size from the median, and the centre indicates the median of the tandem repeat size. c, Rare tandem repeat expansion burden in different genomic features. Red bars indicate significant enrichment in ASD-affected individuals (family-wise error rate; FWER <20%). The horizontal dashed line represents odds ratio=1. An ANOVA test comparing two logistic regression models was used to obtain the results in b and c. d-e, Distance of rare tandem repeat expansions (all individuals), all tandem repeats detected, and known simple sequence repeats to the nearest transcription start site (TSS) (d) and the nearest splice junction (e). Rare tandem repeat expansions (N=258 loci close to TSS and N=297 loci close to splice junctions) are significantly closer to TSS (Wilcoxon test, p=0.01 and 0.003 for all tandem repeats detected (N=5,805 loci) and known simple sequence repeats (N=133,264 loci), respectively) and splice junctions (Wilcoxon test, p=0.03 and 0.002 for all tandem repeats detected (N=7,279 loci) and known simple sequence repeats (N=161,932 loci), respectively). f, Gene set burden analysis of number of rare tandem repeat expansions affecting genes in a gene set comparing ASD-affected individuals (N=1,812) with their unaffected siblings (N=1,485). Orange points indicate odds ratios of gene-sets with FWER <20%. g, Schematic diagram (top) shows the design of a Southern blotting experiment in the targeted tandem repeat in DMPK. Two families with different repeat sizes (1-1039 with expansions and 2-1436 without expansions) are shown. Error bars in a, c and f indicate 95% confidence intervals.

FIG. 4. Clinical analysis of rare tandem repeat expansions in individuals with ASD. a, Comparison of the fraction of samples having rare tandem repeat expansions in females (N=857) versus males (N=4,377) (Fisher's exact test). An odds ratio of more than 1 indicates a higher burden of rare tandem repeat expansions in females. Error bars indicate 95% confidence intervals. b, Comparison of IQ and Vineland Adaptive Behavior standard scores of individuals with (N=139 individuals with IQ score and N=310 individuals with Vineland score) and without (N=426 individuals with IQ score and N=803 individuals with Vineland score) rare tandem repeat expansions (one-sided Wilcoxon test). The minima and maxima indicate 3×inter-quartile range-deviated scores from the median, and the centre indicates the median of the score percentiles.

FIG. 5. Distribution of the number of tandem repeats detected by ExpansionHunter Denovo. The number of tandem repeats detected by ExpansionHunter Denovo in a given sample is stratified by (a) cohort, sequencing platform, and library preparation method. (b) predicted ancestry (from samples in the “MSSNG/Illumina HiSeqX/PCR-free” category in a). Ancestry designations were derived from the 1000 Genomes “super populations” (https://www.internationalgenome.org/category/population): AFR, African; AMR, Admixed American; EAS, East Asian; EUR, European; SAS, South Asian. Other codes: OTH, other; UNK, unknown.

FIG. 6. Number of unique motifs in each repeat-containing region (x-axis).

FIG. 7. Distribution of repeat units (motifs) for the tandem repeats detected by ExpansionHunter Denovo. The 20 most common repeat units are shown.

FIG. 8. Methods for sizing the CGG repeat in LING03. (a) Summary of results on repeat size analyses. PCR-free short-read sequence data with Expansion Hunter not only correctly determined the length of short CGG repeats, but also detected long CGG repeats. We detected the presence of a small deletion adjacent to the repeat in two individuals hindered detection of CGG repeat expansion by Southern blot. (b) The results of PCR amplification of the CGG repeat in LINGO3. Due to the extremely high CG content of the region, long CGG repeats could not be amplified. (c) Sanger sequencing of the PCR-amplified CGG repeat in LINGO3. Note that the bias of PCR towards preferential amplification of shorter amplicons made the chromatogram of longer alleles less prominent. (d) The repeat-primed PCR design and results for the CGG repeat in LINGO3. The predictions for repeat expansions made by Expansion Hunter were consistent with the repeat-primed PCR. Southern blot analysis of the large LINGO3 expansions are shown in FIG. 2f.

FIG. 9. Methods for sizing of the CTG repeat in DMPK. While short CTG repeats were correctly sized by Expansion Hunter (the results were perfectly matched with fragment analysis), slight discrepancies were observed in the estimations for permutation alleles between Expansion Hunter and PCR-based fragment analysis. Note that the length of the premutation CTG repeats (42 CTGs) was close to the read lengths of the HiSeq X platform (150 bp). The predictions of the presence of longer CTG repeats were validated by repeat-primed PCR, although the estimated size by Expansion Hunter was shown to be an underestimation (the saw-tooth pattern of repeat-PCR extended longer than the predicted size).

FIG. 10. Pedigrees of families with rare repeat expansions in FMR1. There is clinical information available for 1-1025-003, 1-1221-003, MSSNG00169-003 and MSSNG00416-003. All of them are diagnosed with Fragile X syndrome.

FIG. 11. Pedigrees of families with rare repeat expansions in DMPK. In the family with clinical information recorded (1-1039), 1-1039-003 was reported to have ASD, myotonic dystrophy, delay development and nocturnal hypoventilation. We experimentally validated no presence of repeat expansion in DMPK in 1-1039-004, AU4076304 and AU4076305. Individual in grey indicates that the corresponding sample is not available for testing.

FIG. 12. Pedigrees of families with rare repeat expansions in FXN. Among families with clinical information recorded (7-0390 and AU0240), 7-0390-003 was reported to have ASD, anxiety, asthma and fine motor delays. AU024004 has been reported to have ASD and athetosis. Individual in grey indicates that the corresponding sample is not available for testing. Motifs of all expanded repeats detected are AAGGAG.

FIG. 13. Pedigrees of families with rare repeat expansions in WWOX. Among families with clinical information recorded (1-0651 and AU3730), 1-0651 family has history of anxiety, schizophrenia and/or other psychotic disorders. 1-0651-003 was reported to have ASD and fine motor delays. AU3730301 was reported to have ASD, ADHD and brain tumor. Individual in grey indicates that the corresponding sample is not available for testing.

FIG. 14. Correlation analysis between parental age and number of repeat expansions detected. An expansion in a child with ASD was defined as de novo if the maximum repeat size of the corresponding parents was below the 75th percentile. Error bars represent 95% confidence intervals.

FIG. 15. Detecting genome-wide tandem repeat expansions in neurological disorders. A) Distribution of repeat unit (motif) sizes for the detected tandem repeats in —300 trio families of epilepsy. The size distribution here is comparable to that in ASD (Trost 2020). B) Rare tandem repeat expansion burden in different genomic features. Red bars indicate significant than expected burden in individuals with epilepsy. More rare tandem repeat expansions are found in intronic regions of epilepsy. FWER: Family-wise error rate.

FIG. 16. Simulation of repeat length distribution when size of tandem repeats are not detected. a) From the output of a tandem repeat detection algorithm, some regions (such as the 1st region shown) would have all tandem repeat sizes detected or genotyped by the algorithm and outliers (circled) are thus identified by the outlier detection approach. b) In the case where the regions (such as the 40,000th region) have some tandem repeat sizes missing from the algorithm, a simulation of normally distributed repeat length is performed in order to identify outliers (circled).

FIG. 17. Pictorial illustration of a method for operating a genetic sequencer.

FIG. 18. Pictorial block diagram illustrative of a computer system.

### DETAILED DESCRIPTION OF THE INVENTION

A method of detecting outlier tandem repeats associated with a disease is provided. The method includes the following steps: i) detecting tandem repeat sequences comprising a repeated motif sequence in nucleic acid samples from individuals within a population of interest which have been sequenced using a sequencing platform, wherein the tandem repeat sequences have a length that permits a tandem repeat calling algorithm to detect and/or genotype the tandem repeat sequences; ii) simulating the length distribution of the tandem repeat sequences in the population of interest to a normal distribution if no tandem repeat sequences are detected in step i); and iii) detecting one or more outlier tandem repeat sequences, wherein an outlier tandem repeat sequence has a length that is greater than that in 90% of the tandem repeat sequences detected in the population interest and occur at a frequency of less than 1% of the tandem repeat sequences detected in a control population.

The term “disease” is used herein to refer to a pathological condition in an individual, including human and non-human mammals. The disease may, for example, be a pathological condition in a developmental process of the central nervous system and brain, heart, kidney, or other organs. Examples of disease conditions thus include, but are not limited to, developmental disorders in any developmental process with respect to the brain, blood, heart, kidney, and others, neurological disease such as neuropathies and neuropsychiatric disorders, cardiomyopathy including congenital heart disease, cancer including carcinomas, sarcomas, lymphomas, gliomas, leukemia, melanomas, etc., diabetes, and others.

The term “neuropsychiatric disorder” is used herein to encompass Autism Spectrum Disorder (ASD), i.e. a disorder that results in developmental delay of an individual such as autism, Asperger's Disorder, Childhood Disintegrative Disorder, Pervasive Developmental Disorder-Not Otherwise Specified (PDD-NOS) and Rett Syndrome (APA DSM-IV 2000); schizophrenia (SZ), intellectual disability (ID), Fragile X syndrome, epilepsy and related nervous system disorders such as OMIM (Online Mendelian Inheritance in Man) nervous system disorders.

The term “population of interest” refers to a population within which individuals may have the disease to be detected, but includes individuals which do not have the disease. Populations may include a particular ethnic population, a population in a particular age group, a population in a given locale, a pediatric population, etc.

The term “control population” refers to a population distinct from the population of interest in which prevalence of the disease to be detected is equal or lower than that in the population of interest.

In the present method, nucleic acid from individuals within a population, e.g. humans, having a disease of interest, is obtained for analysis. Nucleic acid from various biological samples may be studied, depending on the disease of interest, including samples such as blood, serum, plasma, urine, and biopsied tissue from an individual. The sample may be obtained using techniques well-established and known to those of skill in the art, and will vary depending on the sample type as one of skill in the art will appreciate. Examples of different techniques that may be used to obtain a tissue sample include standard biopsy, needle biopsy, endoscopic biopsy, stereotactic biopsy, neuroendoscopy, bone marrow biopsy, and combination techniques which employ biopsy and imaging techniques. Tissue samples from cerebellum, breast, heart, liver, muscle, kidney, thyroid, pancreas, prostate, spleen, and testis may be used. For neurological disease, including neuropsychiatric disorders, preferred biological samples include brain samples, for example, neocortex, amygdala, cerebellar cortex, hippocampus, mediodorsal nucleus of the thalamus, and striatum. For cardiomyopathy, nucleic acid from the heart and related tissue is preferred. The samples may be obtained from any age group, including prenatal, child and adult samples. Generally, a suitable sample will contain up to about 1 μg, typically, in the range of about 50 ng-1 μg of nucleic acid.

Nucleic acid from the tumour sample may be extracted from the sample using techniques well-known to those of skill in the art, including chemical extraction techniques utilizing phenol-chloroform (Sambrook et al., 1989), guanidine-containing solutions, or CTAB-containing buffers. As well, as a matter of convenience, commercial DNA extraction kits are also widely available from laboratory reagent supply companies, including for example, the QIAamp DNA Blood Minikit available from QIAGEN® (Chatsworth, Calif.), or the Extract-N-Amp blood kit available from Sigma-Aldrich® (St. Louis, Mo.).

The nucleic acid samples are sequenced using any appropriate nucleic acid sequencing platform, including but not limited to, including methods such as Next Generation Sequencing (NGS) methods (e.g. such as Illumina, BGI/MGI, Ion torrent and Ion proton sequencing) and Third Generation Sequencing (e.g. such as Oxford Nanopore Technologies and Pacific Biosciences) to permit detection of tandem repeat sequences. Prior to sequencing, the nucleic acid may be amplified using a PCR-based amplification technique.

Tandem repeat sequences are then detected. Tandem repeat sequences are sequences comprising a repeated motif sequence having about 1-25 base pairs which occur directly adjacent to one another, e.g. ACACAC . . . . The repeated motif sequence preferably comprises at least 2 base pairs up to 5, 10, 15 or 20 base pairs. More preferably, the repeat motif sequences are about 2 to 6 base pairs in length. Tandem repeat sequences useful in the present method are those having a length that permits detection and/or genotyping by a tandem repeat calling algorithm. In one embodiment, the tandem repeat sequence length is greater than 150 base pairs when a sequencing platform such as an Illumina-based platform is used. Methods suitable for detecting tandem repeat sequences include, but are not limited to, methods which use anchored in-repeat reads (paired reads in which the first read maps to a repetitive region and the second “anchor” read maps to an adjacent non-repetitive region) to estimate the size and location of genomic repeats. Algorithms that may be used include, but are not limited to, ExpansionHunter Denovo, NanoSatellite, and others.

One or more outlier tandem repeat sequences within the tandem repeat sequences is then detected. Outlier tandem repeat sequences, also referred to herein as “repeat expansions”, are tandem repeat sequences which have a length that is greater than 90% of the lengths of the tandem repeat sequences detected in the population of interest, e.g. a length that is greater than 95% or more than the length of the tandem repeat sequences detected, and preferably occur at very low frequency, for example, less than 1% of the tandem repeat sequences detected in a control population, for example, less than 0.5%, or less than 0.1% of a control population. In embodiments of the present method, outlier tandem repeat sequences associated with disease comprise an increased GC content of at least about 10% in comparison to the tandem repeat sequences detected. In other embodiments, outlier tandem repeat sequences associated with disease are sequences located within intronic regions of a gene, and which may be located close to a transcriptional start site (TSS) or a splice junction, e.g. within about 10,000 base pairs to a TSS or splice junction.

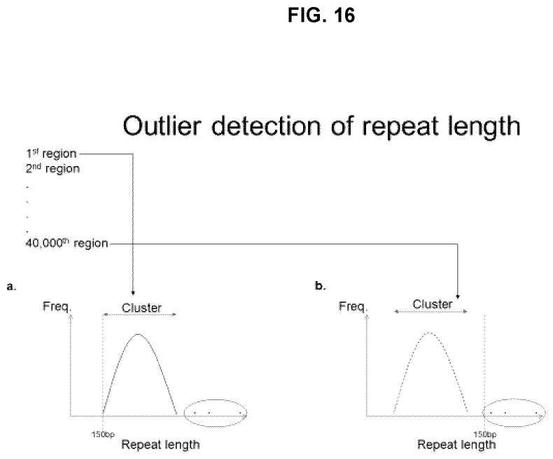

In view of the cut-offs applied in the present method, it is possible that no tandem repeat sequences will be detected by the detection algorithm used. In this case, the length distribution of the missing tandem repeat sequences is adjusted (or simulated) to a normal distribution, for example, with a mean of one, a standard deviation of 0.25, and a maximum of two. This is illustrated in FIG. 16. In the case illustrated in FIG. 16a, the output of a tandem repeat detection algorithm shows that all tandem repeat sizes are detected and/or genotyped by the algorithm (such as ExpansionHunter Denovo) because the tandem repeat sizes are all longer than the sequence read length, 150 bp. The outlier tandem repeat sequences are, thus, identified. However, in the case where tandem repeat sizes are not detected in regions (such as the 40,000th region as shown in FIG. 16b) by the algorithm (e.g., in this case, repeat tandem length is shorter than the sequence read length, 150 bp), a simulation of normally distributed repeat length is performed in order to identify outlier tandem repeat sequences. This normal simulation may be repeated, if required, in order to detect tandem repeat sequences, and the presence of one or more outlier tandem repeat sequences within the population of interest.

Outlier tandem repeat sequences are determined to be associated with a given disease, for example, by functional analysis, including identifying the chromosome location of the outlier tandem repeat sequence, and the gene with which it is associated. Based on this information, the function of the particular gene, the disease with which the outlier tandem repeat sequence is associated may be identified.

The method of detecting outlier tandem repeat sequences, and their association with a disease, is useful to permit diagnosis of the disease in an individual such that the individual may then be appropriately treated.

Thus, in another aspect, a method of diagnosing in an individual risk of having a disease, such as ASD, and optionally treating the individual, is provided. The method comprises the steps of: detecting in a nucleic acid sample from the individual the presence of at least one outlier tandem repeat sequence using a method as described above for detection of outlier tandem repeat sequences. Based on the genomic location of the outlier tandem repeat sequence, diagnosing the individual with the disease associated with that genomic location.

In another embodiment, diagnosis or risk of having a target disease may be based on the detection of a target outlier tandem repeat sequence known to be prominent for the target disease. In this case, a nucleic acid probe that specifically binds to the target outlier tandem repeat sequence, or that binds to a sequence adjacent thereto (a target adjacent sequence), i.e. a sequence that provides selectivity and may be a sequence that is unique with respect to the target outlier tandem repeat sequence. If the target outlier tandem repeat sequence, or target adjacent sequence, is detected in the nucleic acid sample, then the individual is determined to have or be at risk of having the target disease. Following such a determination, a diagnosis may be confirmed using other identifiers of the target disease.

The term “prominent” is used herein to refer to the pronounced presence of the target outlier tandem repeat sequence in the nucleic acid of individuals with the disease than individuals not having the disease. The prominence of the target outlier tandem repeat sequence in individuals with the disease will vary, as one of skill in the art will appreciate. Generally, the target outlier tandem repeat sequence occurs at an increased frequency in a population with the disease than in a control population. For example, target outlier tandem repeat sequences may be present, for example, at least two times more frequently in the ASD population than in the control population. In some cases, the presence of the target outlier tandem repeat sequence may be present at a much greater frequency in a disease population as compared to a control population, e.g. 5-10 times or more frequently in a disease population than in a control population.

Methods of detecting target outlier tandem repeat sequences include those based on the use of primers and/or probes, such as PCR-based techniques and/or Southern blotting techniques, that specifically bind to the target tandem repeat sequence, or to a specific sequence adjacent to the target outlier tandem repeat sequence. Thus, primers/probes will vary with each target outlier tandem repeat sequence and each disease to be diagnosed. Probes will generally be detectably labelled for detection, e.g. with fluorescent, luminescent, radioactive, or other detectable label.

In embodiments, methods of diagnosing ASD is provided in which outlier tandem repeat sequences prominent in some genes are detected, such as LINGO3, DMPK, CACNB1 and FXN. For LINGO3, a CGG outlier tandem repeat is detected; a CTG repeat in DMPK is detected; and tandem repeats with multiple different motifs within the same repeat-containing regions are detected in CACNB1 and FXN, for a diagnosis of ASD in an individual.

Embodiments of the invention are described by reference to the following specific example.

### Example 1

The following study exemplifies methodology in accordance with an aspect of the invention.

### Methods

Samples—Genome sequencing data derived from 8,448 samples from the MSSNG project¹, 9,096 samples from the Simons Simplex Collection (SSC)², and 2,504 samples from the 1000 Genomes Project (1000G)³ was used. All SSC samples used PCR-free DNA library preparation and were sequenced on the Illumina HiSeq X platform (2×150 bp paired-end reads). All 1000G samples used PCR-free library preparation and were sequenced on the Illumina NovaSeq platform (2×150 bp paired-end reads). Each MSSNG sample fell into one of three categories: 1) PCR-based DNA library preparation and sequenced on either the Illumina HiSeq2000 (2×90 bp paired-end reads) or HiSeq2500 (2×126 bp paired-end reads) platforms; 2) PCR-based library preparation and sequenced on the Illumina HiSeq X platform, or 3) PCR-free library preparation and sequenced on the Illumina HiSeq X platform. All samples were aligned to the GRCh38/hg38 reference genome using BWA-mem⁴. Full details on the MSSNG, 1000G, and SSC alignment pipelines can be obtained from the websites of MSSNG, 1000G and SSC (via Globus; https://www.globus.org), respectively.

Genome-wide tandem repeat identification—To perform reference genome-agnostic detection of tandem repeats, ExpansionHunter Denovo (EHdn) (https://github.com/Illumina/ExpansionHunterDenovo), which uses anchored in-repeat reads (paired reads in which the first read maps to a repetitive region and the second “anchor” read maps to an adjacent non-repetitive region) to estimate the size and location of genomic repeats. EHdn v0.7.0 was run on each sample using default parameters. The per-sample output files were combined using the combine_counts.py script provided with EHdn. The final set of regions was generated using the compare_anchored_irrs.py script with the parameter --minCount=2, thus retaining only regions for which at least one sample had C>=2, where C=A*40/R, A is the raw count of anchored in-repeat reads for that region, and R is the average read depth of the sample calculated by EHdn.

Characterization of technical variability in repeat detection—To determine whether the number of repeats estimated by EHdn in a given sample was affected by systematic biases in the sequencing data, the distributions of the raw number of EHdn calls (specifically, the number of regions in the RegionsWithIrrAnchors field of the per-sample JSON output file) were plotted for each combination of cohort (MSSNG, SSC, or 1000G), DNA library preparation method (PCR-based or PCR-free), and sequencing platform (HiSeq 2000/2500, HiSeq X, or NovaSeq).

Sample quality control and ancestry determination—The ancestry of the MSSNG, SSC and 1000G samples was determined using data from 1752 unrelated samples from the 1000 Genomes Project as the reference. The reference samples were genotyped on Illumina HumanOmni2.5-4v1-B and Illumina HumanOmni25M-8v1-1_B chips (http://www.tcag.ca/tools/1000genomes.html). The genotypes for a set of 265,236 autosomal SNPs and 23,171 chromosome X SNPs for the three cohorts with bcftools v1.6 were extracted using the joint-genotyped variant call format (VCF) files as input. For each cohort, the data were sorted, decomposed, normalized and SNVs retained for further processing. The resulting VCFs using PLINK v1.9.b3.42 were formatted. The SNPs with genotyping rate <99% in the reference set or any of the three cohorts were removed. PLINK identity-by-descent estimates were calculated for all pairs of individuals in the three cohorts using the autosomal SNPs to check for pedigree and Mendelian errors within each set and sample duplications between sets. SNPs on X chromosome were used to determine sex and flagged samples where the reported sex and estimated sex were different. Linkage disequilibrium-based pruning of the autosomal SNPs yielded 41,720 SNPs, which were used to estimate model-based ancestry using the program ADMIXTURE′, and projected the three cohorts on the population structure learned from the reference panel.

Validation rate of ExpansionHunter Denovo—To assess the accuracy of tandem repeats identified by EHdn, EHdn was used to detect repeats in the HuRef genome⁶ and then the proportion that could be corroborated by an orthogonal method was determined. Specifically, Illumina HiSeq X reads derived from HuRef blood (NCBI sequence read archive accession number SRR9046649) were aligned to the GRCh38/hg38 reference assembly and EHdn was run as described above. The orthogonal comparison method involved two sources of data: 1) tandem repeats in the human reference genome, derived from tandem repeat finder (TRF)⁷, and 2) insertions and deletions in the HuRef genome detected from Pacific BioSciences single molecule, real-time (SMRT) long-read sequencing data, derived by de novo assembly using Canu⁸ and variant detection using AsmVar (https://github.com/bioinformatics-centre/AsmVar). An EHdn region was considered validated if the sum of the size of the largest overlapping TRF region and the size of overlapping Canu/AsmVar insertions/deletions (positive for insertions and negative for deletions) was at least 150 bp (the minimum size detectable by EHdn). A Canu/AsmVar insertion/deletion was considered to overlap the TRF region if it overlapped the region itself or 100 bp on either side. For example, suppose that a hypothetical EHdn region overlapped a TRF region of size 100 bp, where Canu/AsmVar detected a 70 bp insertion inside the TRF region. The total repeat size would be 170 bp, thereby validating the EHdn call. Conversely, if the TRF region was 160 bp along with a 20 bp Canu/AsmVar deletion, then the total size is 140 bp, and EHdn region would not be considered validated. If the EHdn region did not overlap a TRF region, but there was a Canu/AsmVar insertion 150 bp within the EHdn region, then the EHdn region was considered validated.

Confirmation of repeats detected by ExpansionHunter Denovo—To support the accuracy of EHdn-predicted repeat sizes, the loci listed in Table 2 were genotyped using Expansion Hunter v3.0.2. This program estimates allele-specific repeat size for each genomic coordinate and motif supplied by the user with high accuracy (precision=0.91, recall=0.99). All unique Ehdn-detected repeats (each having a different motif) overlapping each locus were identified. To determine more precise coordinates for input to Expansion Hunter, coordinates from TRF that overlapped the locus were identified. For each combination of TRF coordinates and EHdn motif, Expansion Hunter was used to estimate motif-specific (detected by EHdn) repeat sizes for the samples involved. The Spearman correlation coefficient and p value were then calculated between the EHdn-predicted repeat sizes and the Expansion Hunter estimated size (defined as either the size of the longest allele or the sum of the two allele sizes), aggregated over all of the EHdn-detected motifs for that locus. A manual inspection on the presence of repeat expansion and the corresponding repeat-sequence was performed by inspecting reads from the BAM for repeats found to be expanded by EHdn in Table 2.

Detection of repeat expansions—A repeat expansion was defined as a genomic segment of repeat that is much larger than what is observed in the population. A density-based spatial clustering of applications with noise (DBSCAN) was applied to identify repeat expansions⁹. DBSCAN is a non-parametric clustering algorithm that defines a cluster based on the minimum number of data points (minPts) reachable to each other by a maximum distance (c). Any data point not reachable by the clusters are classified as noisy data or outliers, if they have a value of a particular feature (e.g. size of repeat) higher than those of cluster members. By trial and error, the stringent DBSCAN parameters used in detecting repeat expansions were minPts=−log₂(n)≈14 and ε=2×Mo(Xᵢ), where n is the number of samples, Mo is Mode and Xᵢ is a vector of repeat sizes for repeat i. For a repeat to be detected by EHdn, it must be larger than the sequence read length (e.g., >150 bp). As a result, many samples were left without EHdn's size estimation for the repeat-containing regions that did not meet this size minimum. Similarly, DBSCAN might also fail to detect outliers when only a few samples have genotype data. Therefore, the normalized read depth of the repeat for such samples was simulated by assigning them a normal distribution with a mean of one, a standard deviation of 0.25, and a maximum of two. As a result, a minimum of the normalized read depth of two was required for a repeat to be identified as an expansion.

Experimental validation of repeat expansions—The validation of the repeat length estimated by ExpansionHunter or EHdn was done by fragment analysis with FAM-labelled primers and capillary electrophoresis. PCR was performed with Expand Long Template PCR System™ (Roche), adding dimethylsulfoxide to achieve the final concentration of 5-10%, depending on the GC content of the target region. Capillary electrophoresis with Applied Biosystems' 3730xl™/3130™ capillary sequencers was performed with GeneScan 500LIZ™ size markers. For the CGG repeat in LINGO3, betaine (final concentration: 2 M) was added in the PCR reaction mixtures and the repeat size was determined by Sanger sequencing of PCR products. For samples that appeared to be homozygous for the repeat length, the presence of expanded repeats was validated by repeat-primed PCR (RP-PCR) and/or Southern blot. For RP-PCR, the following repeat-priming primers with the tail sequence of 5′-TACGCATCCCAGTTTGAGACGC-3′ (SEQ ID NO: 1) were used:

CTG-repeat-binding primer:
(SEQ ID NO: 2)
5′TACGCATCCCAGTTTGAGACGCAGCAGCAGCAGCAGCA-3′;
CAG-repeat-binding primer:
(SEQ ID NO: 3)
5′-TACGCATCCCAGTTTGAGACGC TGCTGCTGCTGCTGCT-3′;
CAGG-repeat-binding primer:
(SEQ ID NO: 4)
5′-TACGCATCCGAGTTTGAGACGC CTGCCTGCCTGCCTG-3′;
and
CCG-repeat binding primer:
(SEQ ID NO: 5)
5′-TACGCATCCCAGTTTGAGACGC GGCGGCGGCGG-3′.

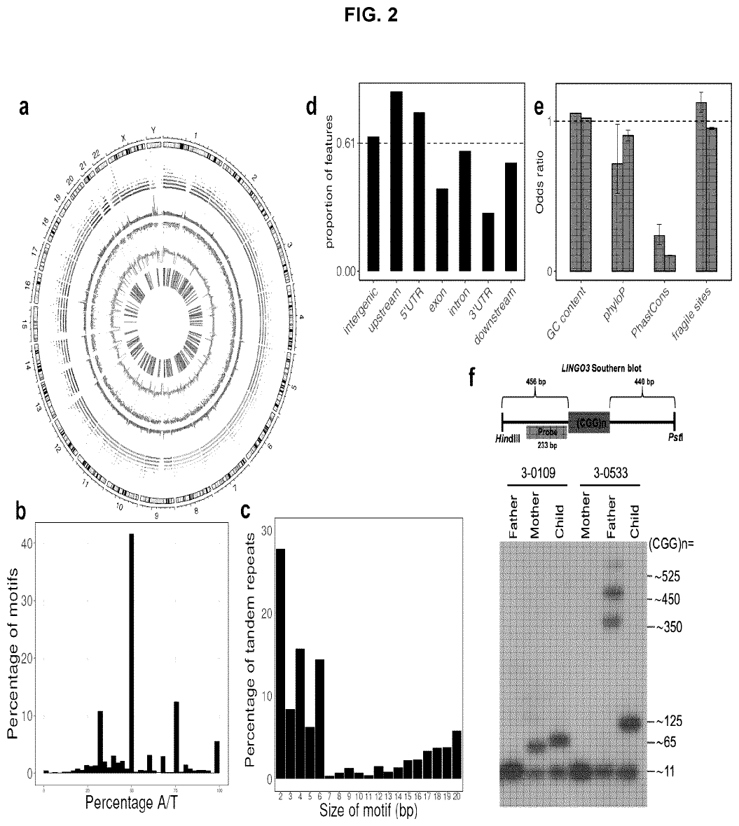

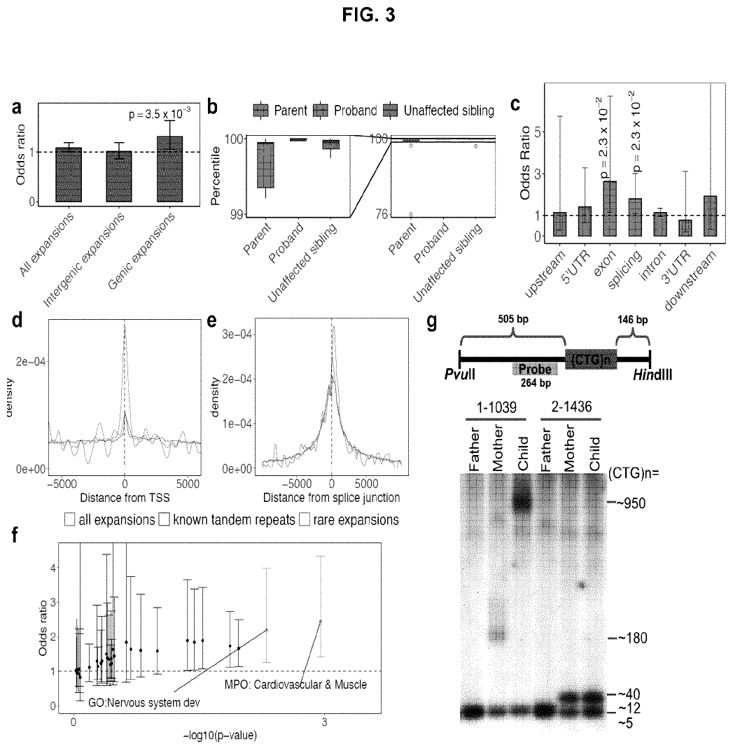

Southern blot was performed to determine the sizes of the CGG repeat in LINGO3 and the CTG repeat in DM1 with selected restriction-endonucleases to digest genomic DNA as denoted in FIG. 2f and FIG. 3g. The DNA fragments were resolved on agarose gels, transferred to membranes and detected by hybridizing radioactive probes specific to DNA sequences adjacent to the repeat tracts. The probes were produced by PCR amplification of the targeted genomic DNA with ³²P-dCTP, and hybridization which permits detection of repeat length heterogeneity. PCR primers to LINGO3 were 5′-GTGTCCGAGGACCTCCTGT-3′ (SEQ ID NO: 6) and 5′-CTCTGAGGGCCACATAAGGA-3′ (SEQ ID NO: 7), and for DMPK were 5′-CGAGTCCCAGGAGCCAATCA-3′ (SEQ ID NO: 8) and 5′-CGGGCACTCAGTCTTCCAAC-3′ (SEQ ID NO: 9).

For repeats that were detected with multiple different motifs at the same repeat-containing regions (e.g., CACNB1 and FXN), Sanger sequencing was performed on the PCR-amplified alleles after gel-extraction to confirm the presence of the reported motifs. PCR primers for CACNB1 were 5′-CTTCCTACCGATTTCCCCTC-3′ (SEQ ID NO: 10) and 5′-CTGATTGACTTCCCACCCTT-3′ (SEQ ID NO: 11) and for FXN were 5′-TATTTGTGTTGCTCTCCGGAG-3′ (SEQ ID NO: 12) and 5′-ATAGTGCACAGAAGCCAAGT-3′ (SEQ ID NO: 13).

Burden analysis of repeat expansions in individuals with ASD—To compare the frequency of rare repeat expansions (<0.1% population frequency) between individuals with and without ASD, a logistic regression analysis was performed by regressing the number of rare repeat expansions on the affected status (unaffected=0, affected=1). Sex bias was avoided by performing the test only on autosomal regions. Any biases in number of repeats detected per subject that may have been related to ethnicity were accounted for. From five admixture variables obtained in the ancestry determination step (see “ancestry determination” above), we included as covariates in the final model only two variables that showed significant correlation with the number of EHdn detected repeats (p<0.05).

Besides this burden test for total number of rare expansions, a functional burden test, as well as a gene set burden test, were performed. The bias in number of rare repeat expansions per subject in functional and gene set burden tests by covarying the number of rare repeat expansions found in intergenic region were further accounted for. For the functional burden test, the genome (RefSeq hg38) was separated into different functional elements, i.e., upstream (1 kb upstream of transcription start sites), 5′UTR, exon, core splice site, intron, 3′UTR and downstream (1 kb downstream of the transcription termination sites). The number of rare repeat expansions impacting each functional element were tested for. If any rare repeat expansion impacted more than one functional element, the effects were prioritized based on their impact on the corresponding genes predicted by ANNOVAR¹⁰. We also tested these different functional elements altogether as a genic burden signal. For the gene set burden test, we obtained 33 functional gene sets previously used to study CNV and SNV enrichment in ASD, including genes relevant to neuronal functions, synaptic components, or genes with homologues in mouse genes grouped by organ system (Table 3). Finally, we estimated family-wise error rate (FWER) to adjust for multiple comparisons.

TABLE 3
Summary of gene sets used for functional burden analysis.

| GeneSet ID (Suppl | geneset | |
|---|---|---|
| DataSets) name | number of | genes |
| Neurof_PathwaysAxonG | Axon guidance pathways | 388 |
| Neurof_KeggSynaptic | KEGG synaptic pathways | 407 |
| Neurof_GoNeuronBody | GO neuron body | 309 |
| Neurof_GoSynaptic | GO synapsis | 622 |
| Neurof_GoNeuronProj | GO neuron projection | 1230 |
| Neurof_GoNervTransm | GO neurotransmission | 716 |
| Neurof_GoNervSysDev_CNS | GO central nervous system development | 774 |
| Neurof_GoNervSysDev | GO nervous system development | 1874 |
| Neurof_UnionInclusive | Neurofunction union inclusive | 2874 |
| Neurof_UnionStringent | Neurofunction union stringent | 1424 |
| FMR1_Targets_Darnell | FMR1 targets Darnell et al | 840 |
| FMR1_Targets_Ascano | FMR1 targets Ascano etal | 927 |
| PSD_BayesGrant_fullset | Post-synaptic density components (Bayes et al, full list) | 1407 |
| PhHs_NervSys_All | HPO: Nervous system, any inheritance | 1590 |
| PhHs_NervSys_ADX | HPO: Nervous system, AD or X-linked | 651 |
| PhHs_MindFun_All | HPO: Higher mental function abnormality, any inheritance | 439 |
| PhHs_MindFun_ADX | HPO: Higher mental function abnormality, AD or X-linked | 153 |
| PhMm_NeuroBehav_all | MPO: Neurological abnormality or abnormal behavior | 2123 |
| PhMm_NervSystem_all | MPO: Nervous system abnormality | 2375 |
| PhMm_NeuroUnion_all | MPO: Neurological abnormality or abnormal behavior or nervous system abnormality | 3202 |
| PhMm_Aggr_ IntegAdipPigm_all | MPO: Adipose or integument or pigmentation abnormality | 1624 |
| PhMm_Aggr_ EndoExocrRepr_all | MPO: Endo- or exocrine or reproductive system abnormality | 2026 |
| PhMm_Aggr_ HematoImmune_all | MPO: Hematological or immune abnormality | 2605 |
| PhMm_Aggr_ DigestHepato_all | MPO: Digestive or hepatobiliary abnormality | 1493 |
| PhMm_Aggr_ CardvascMuscle_all | MPO: Cardiovascular or muscle abnormality | 2059 |
| PhMm_Aggr_Sensory_all | MPO: Sensory system abnormality | 1293 |
| PhMm_Aggr_ SkeCranioLimbs_all | MPO: Skeletal or limb or cranium abnormality | 1588 |
| BSpan_VH_thr4.74 | Brain very high expr | 4600 |
| BSpan_HM_thr3.21 | Brain high/medium expr | 4605 |
| BSpan_ML_thr0.93 | Brain medium/low expr | 4596 |
| BSpan_Ab_thr.MIN | Brain low/absent expr | 4601 |
| Blue_module | Brain specific protein expression | 2484 |
| gnomAD_oe_lof_upper_0.35 | gnomAD LoF intorelance | 2925 |

Statistical comparisons of means—We performed non-parametric Wilcoxon signed-rank tests (one-sided) to compare means between two datasets. These included testing the hypotheses of (i) shorter distances to TSS or splice junction for rare repeat expansions than two other sets of repeats separately (known simple sequence repeats and all EHdn-detected repeats), (ii) lower phenotype-related test scores for samples with than without rare repeat expansions, and (iii) higher number of rare repeat expansions found in affected and unaffected children. For (i), we only included the tandem repeats within 10 kb distance from TSS or splice junction in the test. The distance was calculated from the midpoint of a tandem repeat region to the nearest TSS or splice junction. For (ii), we compared the test scores of the two phenotypes (Vineland Adaptive Behavior standard score to measure adaptive function, and IQ full scale standard score to measure cognitive ability) available in MSSNG database's samples with (n=2,417) and without (n=1,927) rare repeat expansions. This is to test if a similar reduced adaptive function or cognitive ability can be found in individuals with rare repeat expansions, as we previously showed such phenomenon in the carriers of rare pathogenic SNVs or CNVs. Samples included were mutually exclusive to each other and there were no replicates (randomization not applicable).

### Results

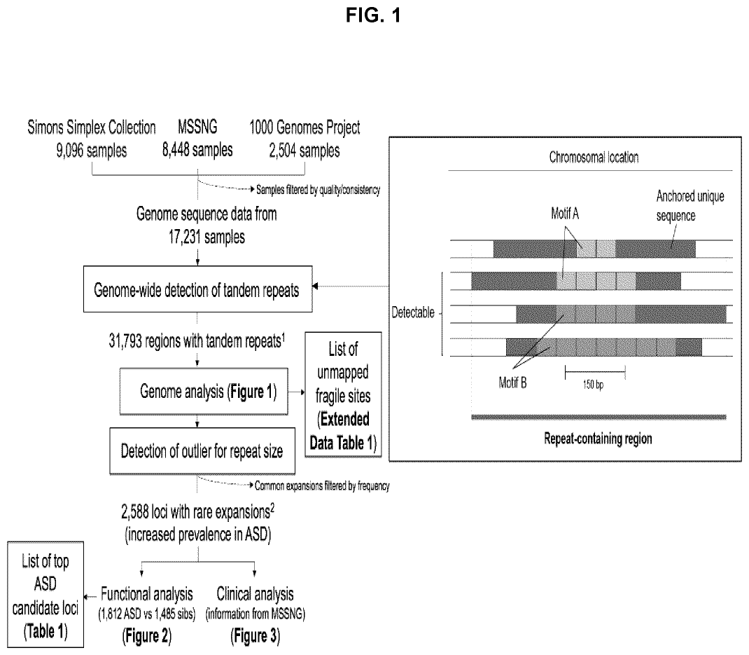

Detection of tandem repeats from genome sequence data—To assess the characteristics of tandem repeat expansions in the human genome, data was collected from 20,048 genomic samples sequenced on Illumina platforms with >30× coverage. These consisted of (a) 8,448 samples (2,042 families with both parents and at least one child, and 1,844 singletons) from families with ASD from the Autism Speaks MSSNG project, (b) 9,096 samples (1,941 complete quartet families) from ASD families from the Simons Simplex Collection (SSC), and (c) 2,504 samples of unrelated population controls from the 1000 Genomes Project. All genomes were aligned to the GRCh38/hg38 reference assembly. We estimated the length of tandem repeats using the ExpansionHunter Denovo (EHdn) algorithm². EHdn detects any tandem repeat involving a motif of 2 to 20 bp, for which the total size is larger than the sequencing read length (e.g., 150 bp for Illumina HiSeq X) (FIG. 1). It functions irrespective of prior knowledge of the presence or expected sequence of the tandem repeats at any given region. The criteria that EHdn uses to identify tandem repeats are detailed in the Methods.

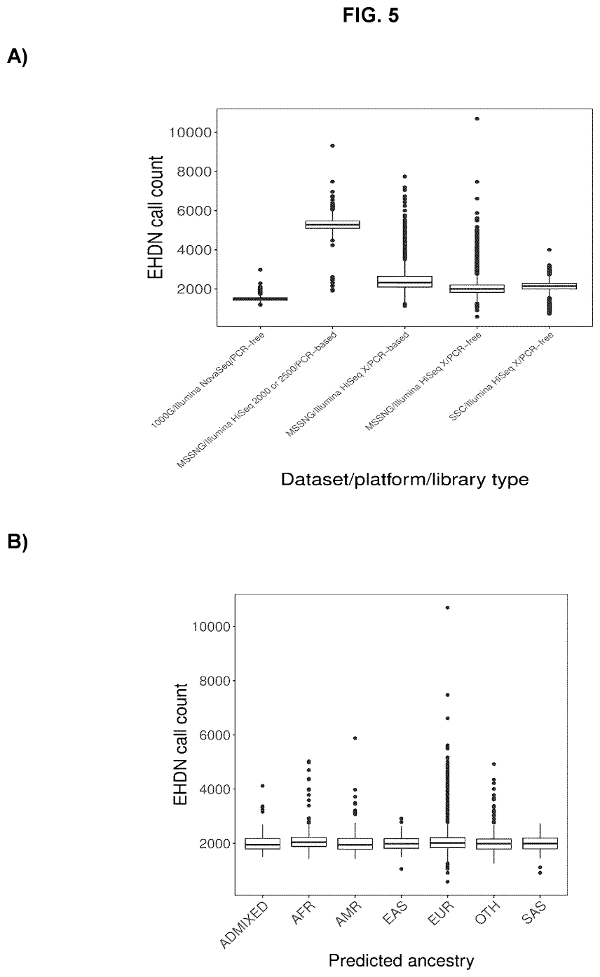

77% of the repeats detected by EHdn in the HuRef genome were validated by comparing to repeats detected by an orthogonal strategy of long-read sequencing (PacBio) data (Methods). Samples were appropriately curated (FIG. 5). 36 samples with Mendelian error or sex mismatch were removed. It was found that samples sequenced from PCR-based libraries and/or using the Illumina HiSeq 2000/2500 platforms had higher numbers of apparent tandem repeats detected per sample (FIG. 5). Since such samples are likely driven by PCR's stutter artifact or differences in sequencing technology, these were removed from subsequent analyses. Similarly, 243 samples from cell line-derived DNA were removed for consistency of DNA source (i.e., only whole blood-extracted DNA was used). In addition, 74 samples for which the total tandem repeat counts were >3 standard deviations were removed from the overall mean number of repeats detected in order to avoid bias in the outlier detection (Methods). For multiplex families, we retained only one affected child per family (specifically, we kept the individual with earliest sample ID). We thus had a final set of 17,404 genomic samples (3,699 ASD families, 1,550 ASD singletons and 2,504 controls).

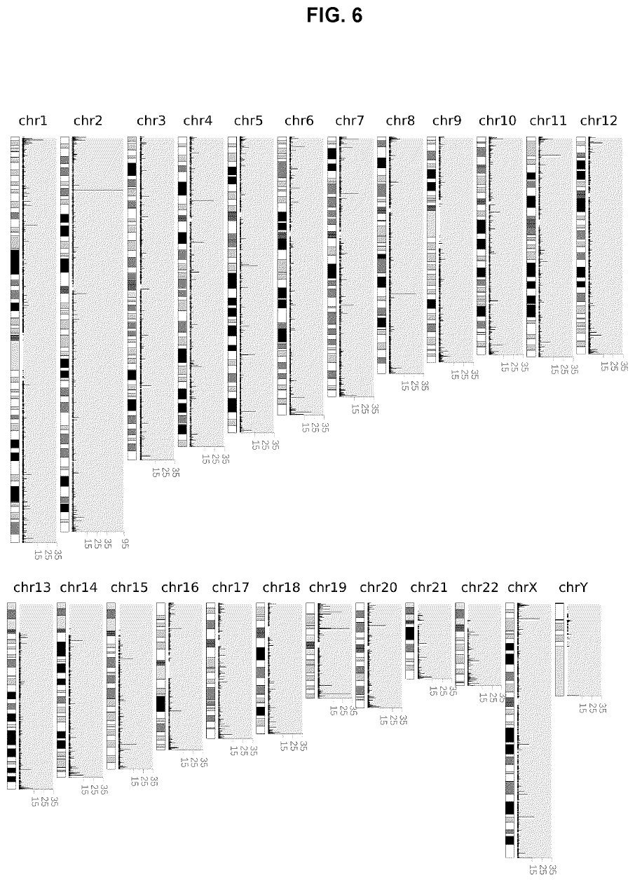

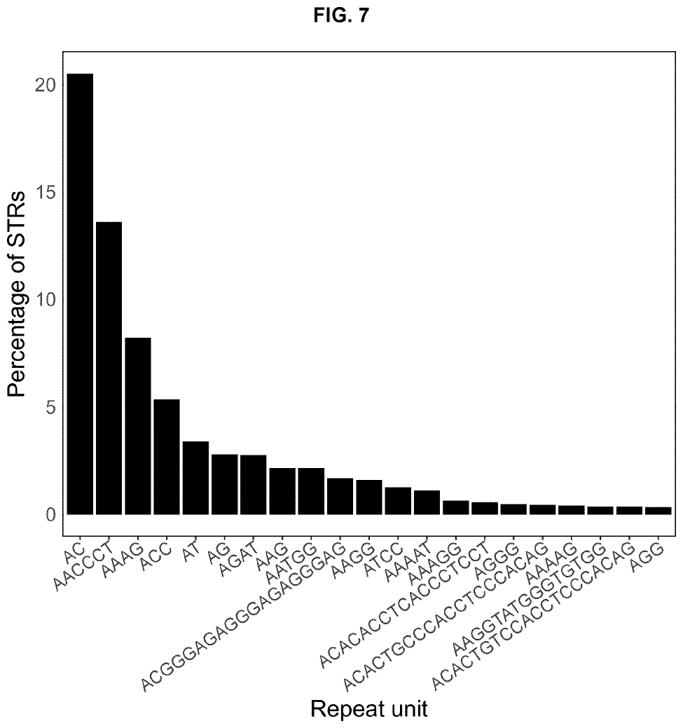

Wide variability of repeat motif size, genic context, and fragile sites—39,078 repeat motifs in 33,083 distinct regions of the human genome were identified, revealing that a given tract could have more than one repeat, with ˜1.2 different forms of repeat-sequence per region. We defined a tandem repeat-containing region as a genomic location where the repeats detected with one or more different motifs overlapped with each other by at least 1 bp (FIG. 1). The number of motifs per region varied across chromosomes in an apparently non-random manner (FIG. 2a and FIG. 6). There were 2,537 regions (7.7%) with more than one motif, and as many as 92 different motifs in a single region (chr2:32915989-32916586; FIG. 6). The motifs were predominantly (>40%) AC- (or GT- on the opposite strand) or AG- (or CT- on the opposite strand) rich (FIG. 2b) (FIG. 7). Of the motifs, 5.4% were composed only of A or T nucleotides, and only 0.38% were of C or G only (FIG. 2b). In terms of motif size, the majority (72.4%) were <7 bp, even though EHdn could detect motifs of up to 20 bp. The most common motif size was 2 bp, found in 26.9%. In the smallest size range, even-numbered motif sizes (2, 4 and 6 bp) notably outnumbered the odd-numbered motif sizes (3 and 5 bp), whereas no such trend was evident among the larger motifs (FIG. 2c).

To understand the distribution of the tandem repeat-containing regions, we correlated their presence with different genomic features (FIG. 2d). As expected, they were prevalent in known polymorphic simple sequence repeat regions previously detected by Tandem Repeat Finder in 1,031,708 locations in the human reference genome (odds ratio (OR)=2.0; p<2.2×10⁻¹⁶). Of the repeat-containing regions reported here, 14,003 (42.3%) have not been previously reported. For the repeat-containing regions that overlapped known simple sequence repeat regions, 1,150 (6%) had at least one motif not found in the reference sequence. Further, these repeat-containing regions were more prevalent in GC-rich regions (OR=1.03; p<2.2×10⁴⁶) and all (common and rare) known fragile site regions (OR=1.9; p<9.4×10⁻¹⁹), but relatively depleted within conserved DNA sequences (OR=0.86; p<2.9×10⁻⁴ for PhastCons and OR=0.97; p=0.65 for phyloP) (FIG. 2e). In genic regions, repeat-containing regions were more common than the genomic average in upstream (OR=1.4; p<2.2×10⁴⁶) (1 kb from transcription start sites) and 5′ untranslated regions (OR=1.24; p=1.7×10⁻⁵), while less common in exonic (OR=0.65; p<2.2×10⁻¹⁶) and 3′ untranslated regions (OR=0.46; p<2.2×10⁻¹⁶) (each feature was normalized by its corresponding size spanning across the genome) (FIG. 2d).

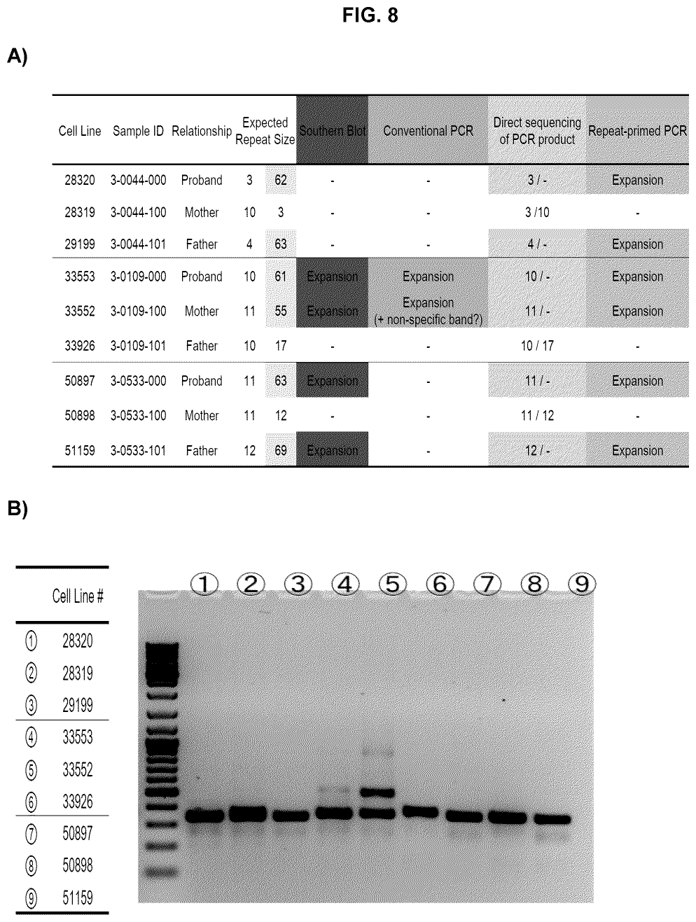

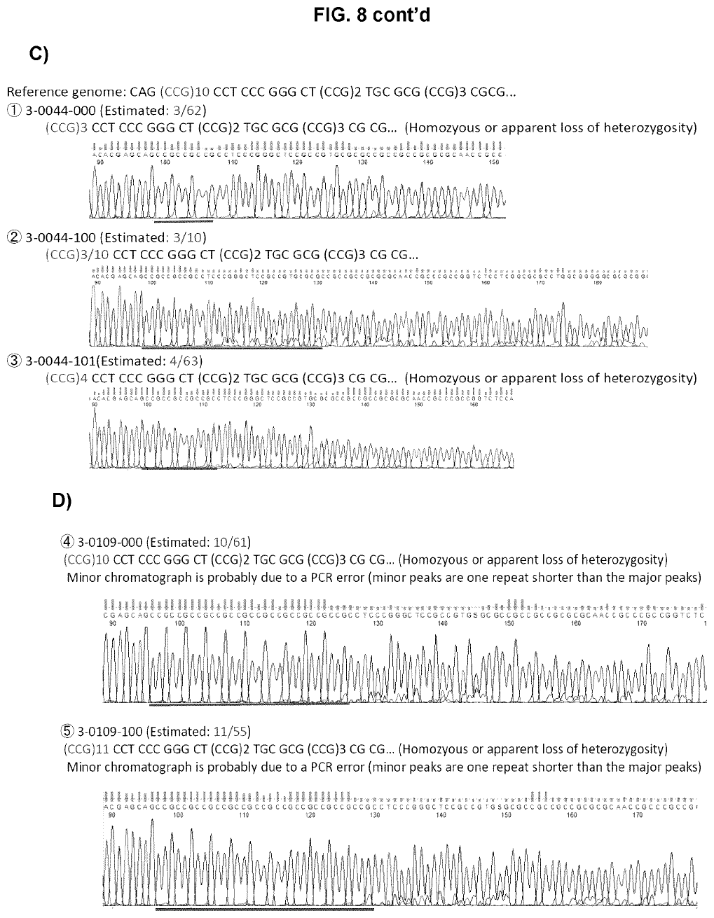

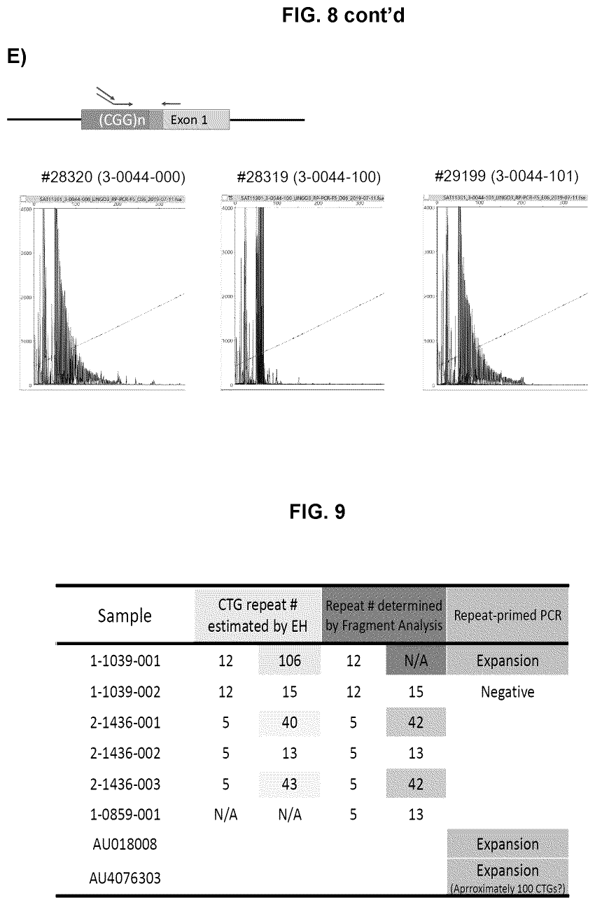

The increased recognition of repeats in cytogenetically known fragile site locations may allow refined mapping of those that are not yet characterized at molecular resolution, and provide important information on susceptibility to genome instability. Indeed, repeat-containing regions we identified co-localized to 9 of 13 (69.2%) of the molecularly mapped rare folate-sensitive fragile sites, all at CG-containing repeats, including the cytogenetically confirmed FRA12A/DIP2B, FRAXA/FMR1, and FRAXE/AFF2. Intriguingly, 66.7% (10 of 15) of the currently molecularly unmapped fragile sites overlapped with at least one GC-rich tandem repeat-containing region detected (Table 1). One of the potentially novel mapped fragile sites was FRA19B, which overlapped with a CGG repeat detected at the 5′ untranslated region in LINGO3. Expansion in one such available sample was confirmed by repeat-primed PCR and Southern blotting (FIG. 2f). Other examples can be found in FIG. 8.

TABLE 1
Molecularly unmapped rare folate-sensitive fragile
sites overlapped with GC-rich tandem repeats.

| Site | Location | Motif | Coordinate | Gene(s) |
|---|---|---|---|---|
| FRA1M | 1p21.3 | CCG | chr1:94418144-94418774 | ABCD3 |
| FRA2L | 2p11.2 | CCG | chr2:86914283-86915185 | RGPD1 |
| FRA2B | 2q13 | CCG | chr2:111120478-111121517 | BCL2L11 |
| FRA2K | 2q22.3 | CCG | chr2:147844114-147844677 | ACVR2A |
| FRA5G | 5q35 | CCG | chr5:177553859-177554905 | FAM193B |
| FRA8A | 8q22.3 | CCCGCCGCCG | chr8:101205417-101205854 | ZNF706 |
| | | CCGCGCG | | |
| | | CCG | chr8:103298253-103299692 | BAALC-AS1/FZD6 |
| | | CCG | chr8:104588661-104589187 | LRP12 |
| FRA12D | 12q24.13 | CCG | chr12:112381861-112382745 | HECTD4 |
| FRA19B | 19p13 | CCCCGCG | chr19:892485-893322 | MED16 |
| | | CCG | chr19:2307584-2308627 | LINGO3 |
| | | CCG | chr19:2311527-2311819 | |
| | | CCCCCCCCCCC | chr19:2436121-2437305 | LMNB2 |
| | | CCCCCCCCG | | |
| | | CCG | chr19:10870902-10872011 | CHARM1 |
| | | CCG | chr19:15332062-15332900 | BRD4 |
| | | CCG | chr19:14495496-14496620 | GIPC1 |
| FRA20A | 20p11.23 | CCG | chr20:20678904-20679127 | RALGAPA2 |
| | | CCG | chr20:20712192-20712984 | |
| | | CCG | chr20:20714199-20714410 | |
| FRA22A | 22q13 | CCG | chr22:38316846-38317855 | CSNK1E |

Rare repeat expansions in ASD individuals, in genes related to nervous system, cardiovascular system and muscle—Repeat expansions that are disease-causing and functionally impactful tend to be large and rare in the general population. We applied a non-parametric approach to identify individual repeats whose tract lengths were outliers compared to those among members of the cohorts (Methods). We designated these outliers as repeat expansions. We defined these as having at least double the tract length of that in the majority of the samples (>2 times the length of the mode) and not being a member of any clusters in the size distribution (Methods). While our defined criterion of an expansion is a conservative measure, it should be noted that there are diseases whose repeat tracts incurred one or two additional repeat units that lead to disease. Such changes will be missed by EHdn. We further categorized them as rare repeat expansions when found in <0.1% of the population controls (1000 Genomes Project). This resulted in 2,483 repeat-containing regions (3,818 motifs) being categorized as rare repeat expansions (FIG. 1).

To delineate the possible functional roles, we assessed whether the rare repeat expansions identified here contribute to the risk of ASD and its heritable features. To avoid sex bias on allele transmission, we compared their occurrence only in autosomal regions, in children with (N=5,249) and without (N=2,023) ASD from 5,262 unrelated families. We further adjusted the comparisons by adding ethnicity as covariates in the statistical tests (Methods). We found that rare repeat expansions were more prevalent in children with ASD than in the non-ASD siblings (OR=1.2, p=8×10⁻⁶). These rare repeat expansions generally represented further expansions from already-large repeat expansions from the previous generation, since the average repeat-tract length of these parents was at the 94th percentile of the length distribution (FIG. 3a). This transmission expansion bias of longer repeats is consistent with the instability trends for almost every disease-associated repeat. To account for batch effects across families and cohorts, we compared the occurrence of rare repeat expansions within genic regions (as defined above) in the children, and added the number of rare repeat expansions in intergenic regions as a covariate in the statistical comparison. We found that rare repeat expansions were consistently more prevalent in children with ASD than controls (OR=1.24, p=0.0002) (FIG. 3b). A similar increase among ASD-affected children was observed when we compared them in the SSC cohort alone (OR=1.26, p=0.0004). The detection rate of rare repeat expansions was 35.3% in ASD children and 31.3% in unaffected children. From this difference, we estimated that as much as 4% of the ASD risk may be contributed by rare repeat expansions (Wilcoxon's test, p=0.001).

Towards assessing possible functional effects of the rare repeat expansions, we examined their proximity to different features within genes. We found the ASD-associated rare repeat expansions to be increased in exonic (OR=2.58, p=0.0002, family-wise error rate; FWER=0.02), intronic (OR=2.21, p=0.005; FWER=0.04) and splicing (OR=1.68, p=0.007; FWER=0.05) regions (FIG. 3c). The proximity to genes for the ASD-associated repeat expansions may suggest their regulatory roles. For example, compared to the known simple sequence repeats and EHdn-detected repeats, we found that rare repeat expansions were located closer to the genes' nearest transcriptional start sites (Wilcoxon's test, p=0.0001 and 0.0015 for known simple sequence repeats and EHdn-detected repeats, respectively) and splice junctions (Wilcoxon's test, p=0.0002 and 0.007 for known simple sequence repeats and EHdn-detected repeats, respectively) (FIG. 3d-e).

In terms of the biological pathways associated with the genes impacted by the identified rare repeat expansions, we investigated their relevance to previously known ASD-related gene functions and pathways using the pathway enrichment test (Methods). Unlike rare SNVs or CNVs, which predominantly impact neural synaptic functions, ASD-linked genes with rare repeat expansions were predominantly involved in nervous system (OR=1.76, p=0.002; FWER=0.06), and cardiovascular system or muscle (OR=1.55, p=0.005; FWER=0.16) (FIG. 3f). One example was the CTG repeat found in DMPK, whose expansion to greater than 50 units is known to cause myotonic dystrophy type 1 (DM1) (OMIM ID: 160900) (FIG. 3g). Approximately, 5% of individuals with DM1 also have ASD. We identified 8 individuals with ASD from 7 unrelated families and 1 individual without ASD (OR=2.7, p=0.46) that carry rare CTG repeat expansions in DMPK (experimentally validated with repeat-primed PCR and Southern blotting; FIG. 3g and FIG. 9). In these cases, our independent, unbiased approach confirmed a previously indicated comorbidity of ASD with DM1.

From the gene sets that were enriched in the pathway enrichment test, we selected 12 other examples (repeats at FGF14, CACNB1, FXN, CDON, MYCOD, WWOX, PARD3, IGF1, FOXJ3, ABCC4, RICTOR and ARID1B) (OR>1.5 each with at least 4 unrelated ASD carriers) as top candidates to be ASD-relevant repeat-containing regions when expanded (Table 2). We further genotyped each of these 13 genes, confirming by another tandem repeat detection algorithm, Expansion Hunter. Although not included in the prior statistical comparisons, known ASD-risk regions such as the CGG repeats at the 5′ untranslated regions of FMK1 and AFF2 were among the top loci, based on the same criteria (Table 2). Due to their rarity, none of these regions considered individually were statistically increased in ASD subjects; however, rare repeat expansions in 176 loci within these enriched gene sets collectively accounted for 5.5% (288 of 5,249) of the ASD cases in the cohorts (OR=1.49, p=0.0014).

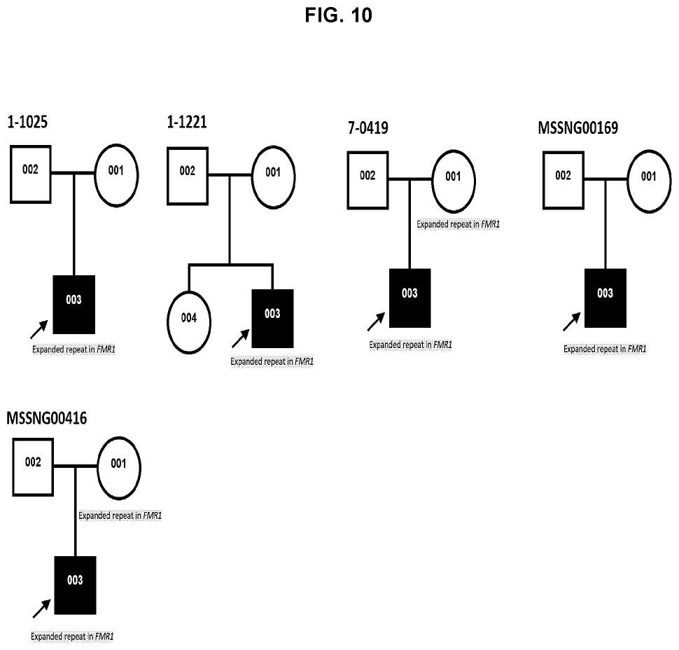

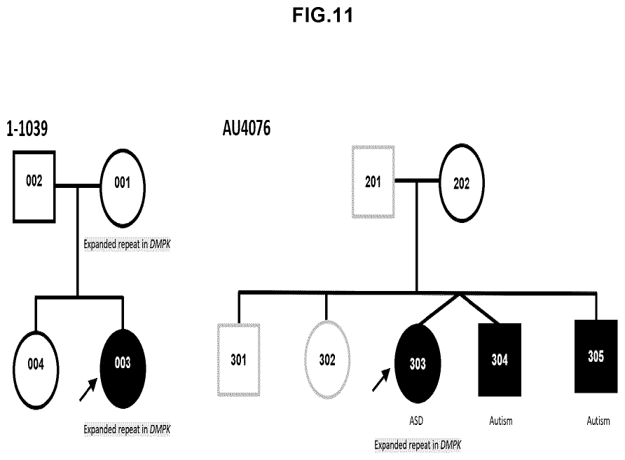

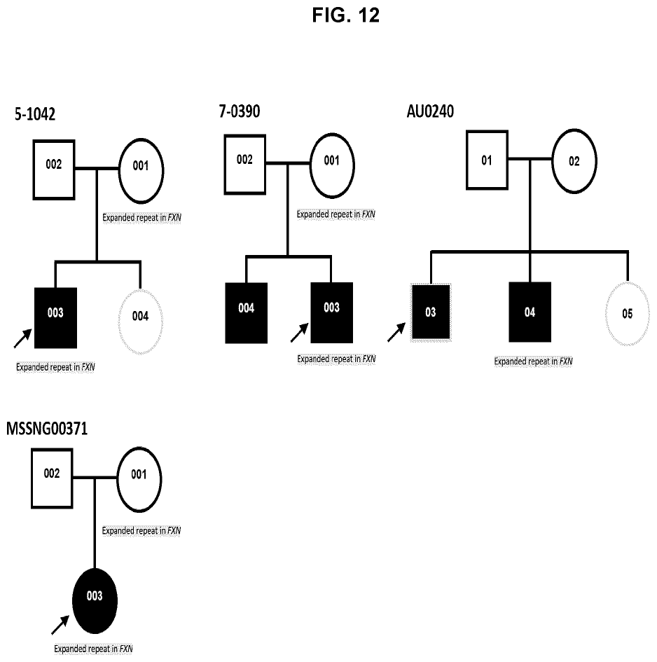

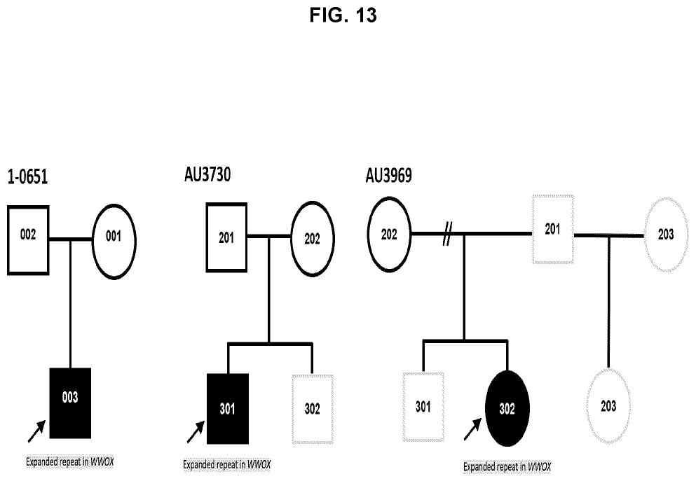

Individuals with rare repeat expansions correlate with ASD-related phenotypes—Towards correlating the genetic findings herein with the phenotypes in the MSSNG cohort, we note that all 4 males with clinical information available in the database with CGG repeat expansions in FMR1 were indicated as having fragile X syndrome (FIG. 10). Similarly, the proband (family 1-1039) with a rare repeat expansion of (CTG)_{˜950} detected in DMPK was reported as having DM1 and other developmental problems (FIG. 11) (for another individual's data in MSSNG is not yet available). Her mother, who carries a repeat of (CTG)_{˜180} in DMPK (FIG. 3g), also reported history of difficulties in motor coordination (i.e., genetic anticipation). Examples of pedigrees with individuals detected with expanded repeats at other genes (FXN and WWOX) are presented in FIGS. 12 and 13.

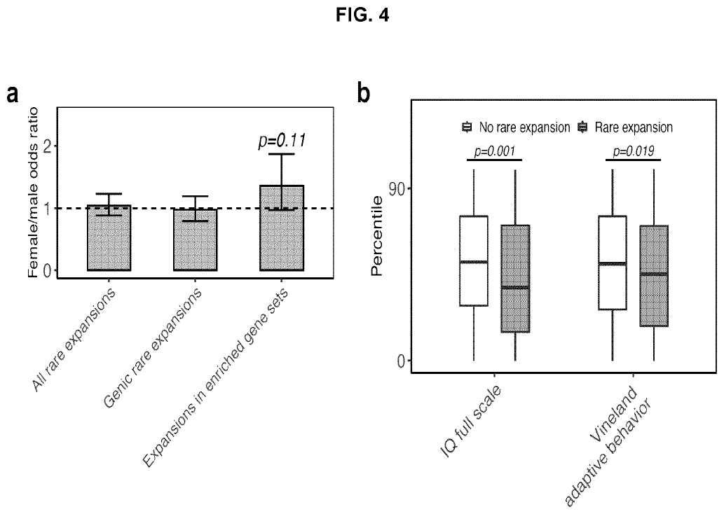

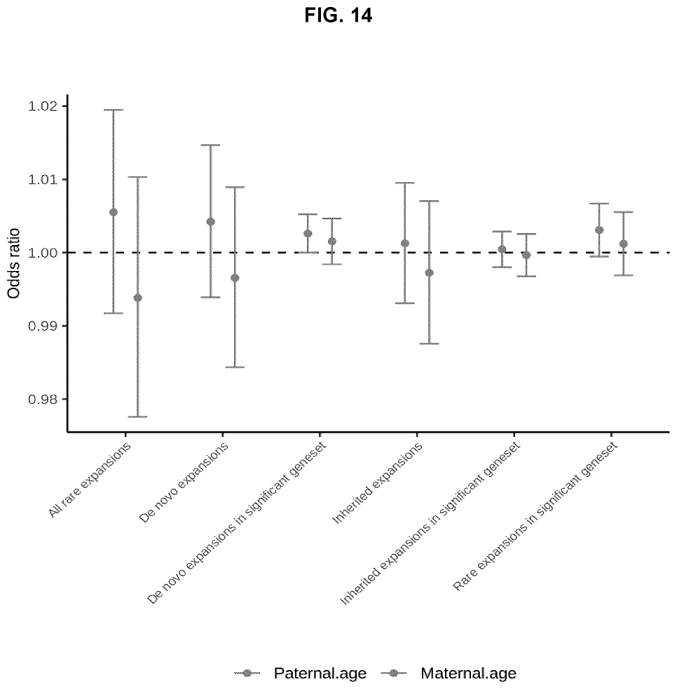

As with the carriers of de novo loss-of-function SNVs or CNVs, we found rare repeat expansions in the enriched gene sets more often in females than in males (OR=1.46; p=0.01) (FIG. 4a), further supporting the differential genetic loading for males and females in ASD″. Although not statistically significant, there was a trend of more rare repeat expansions detected in children with older fathers (FIG. 14). Consistent with our previous findings for rare pathogenic SNVs and CNVs, subjects with rare repeat expansions had lower IQ (Wilcoxon's test, p=0.003) and Vineland Adaptive Behavioral standard scores (Wilcoxon's test, p=0.022). This provides compelling evidence for the role of rare repeat expansions in ASD-related phenotypes.

TABLE 2
Top candidate ASD-relevant repeat loci

| | | | | | | | | | Known | Po- | |
|---|---|---|---|---|---|---|---|---|---|---|---|
| | | | | | | | o/e | Known | disease- | tential | |
| | Major | Risk | # of | | | Genic | con- | ASD | linked | fragile | |
| Coordinate¹ | motif | motif(s) | cases | OR | Gene | region | straint | gene | expansion | site | OMIM disorder |
| chr13: | AAG | AAGGAG:AAGA | 9 | 3.5 | FGF14 | intronic | 0.39 | Novel | Novel | FRA13D | Spinocerebellar |
| 102160822- | | GG:AAAGAAGA | | | | | | | | | ataxia |
| 102162469 | | AG:AAGAAGCA | | | | | | | | | type 27 |
| | | G | | | | | | | | | |
| chr17: | AAG | AAGGAGGAG:A | 8 | 3.1 | CACNB1 | intronic | 0.38 | Novel | Novel | FRA17D | NA |
| 39182673- | | AGAAGGAG:AA | | | | | | | | | |
| 39183931 | | GAAGAGGAGG | | | | | | | | | |
| chr9: | AAG | AAG:AAGGAG | 8 | 3.1 | FXN | intronic | 0.72 | Novel | Known | FRA9K | Friedreich’s |
| 69036648- | | | | | | | | | | | ataxia |
| 69037984 | | | | | | | | | | | |
| chr11: | AAGAGGTG | AAGAGGTGGC | 7 | 2.7 | CDCN | upstream | 0.78 | Novel | Novel | FRA11G | Holo- |
| 126063945- | ATAGTATT | AGTATT | | | | | | | | | prosencephaly |
| 126066092 | | | | | | | | | | | |
| chr17: | AAAAT | AAAAT | 7 | 2.7 | MYOCD | intronic | 0.39 | Novel | Novel | FRA17A | NA |
| 12693129- | | | | | | | | | | | |
| 12694105 | | | | | | | | | | | |
| chr19: | AGC | AGC | 7 | 2.7 | DMPK | 3′UTR | 0.51 | Known | Known | NA | Myotonic |
| 45769551- | | | | | | | | | | | dystrophy type 1 |
| 45770697 | | | | | | | | | | | |
| chrX: | CCG | CCG | 7 | Inf | FMR1 | 5′UTR | 0.42 | Known | Known | FRAXA, | Fragile X |
| 147911368- | | | | | | | | | | mapped | syndrome |
| 147912629 | | | | | | | | | | | |
| chrX: | AT | ACACATATGTA | 7 | 2.7 | IL1RAPL1 | intronic | 0.2 | Known | Novel | FRAXL | Mental |
| 29802527- | | TACATGTAT:A | | | | | | | | | retardation, |
| 29803810 | | CACATATGTAT | | | | | | | | | X-linked |
| | | ATATGTAT | | | | | | | | | |
| chr16: | AAAAG | AAAAG | 6 | Inf | WWOX | intronic | 1.53 | Known | Novel | FRA16D | Epileptic |
| 78722789- | | | | | | | | | | | encephalopathy, |
| 78724123 | | | | | | | | | | | early infantile |
| chr10: | AACAG | AACAG:AACAG | 5 | 1.9 | PARD3 | intronic | 0.31 | Novel | Novel | FRA10J | NA |
| 34665217- | | G:AACGGG | | | | | | | | | |
| 34666477 | | | | | | | | | | | |
| chr12: | AAG | AAGGAG:AAGA | 5 | 1.9 | IGF1 | intronic | 0.81 | Novel | Novel | FRA12L | Insulin-like |
| 102440998- | | GG | | | | | | | | | growth factor I |
| 102442508 | | | | | | | | | | | deficiency |
| chr1: | AAG | AAGGAGGAG:A | 4 | 1.5 | FOXJ3 | intronic | 0.27 | Novel | Novel | FRA1B | NA |
| 42221409- | | AGACG | | | | | | | | | |
| 42222681 | | | | | | | | | | | |
| chr13: | AAGG | AAAG | 4 | 1.5 | ABCC4 | intronic | 0.49 | Novel | Novel | FRA13D | NA |
| 95243999- | | | | | | | | | | | |
| 95245103 | | | | | | | | | | | |
| chr5: | ATATATAT | ATATATATC:A | 4 | 1.5 | RICTOR | intronic | 0.15 | Novel | Novel | FRA5A | NA |
| 38990137- | C | TATATATC | | | | | | | | | |
| 38991105 | | | | | | | | | | | |
| chr6: | AAG | AAG | 4 | 1.5 | ARID1B | intronic | 0.1 | Known | Novel | FRA6M/E | Coffin-Siris |
| 156896665- | | | | | | | | | | | syndrome |
| 156897624 | | | | | | | | | | | |
| chrX: | CCG | CCG | 4 | Inf | AFF2 | 5′UTR | 0.28 | Known | Known | FRAXE, | Mental |
| 148500008- | | | | | | | | | | mapped | retardation, |
| 148501283 | | | | | | | | | | | X-linked |

¹Loci on the X chromosome were not included in the overall statistical comparisons for functional analysis. They were added here only for reference.
OR: odds ratio,
o/e constraint: upper bound observed over expected constraint score from gnomAD.

### Discussion

It is demonstrated that large-scale profiling of repeat expansions from genome sequence data can delineate an unprecedented variability of tandem repeats in the human genome. Specifically, we found 176 tandem repeat loci to be expanded in 288 of 5,249 individuals (5.5%) with ASD, and propose that such expansions may be relevant to ASD. Our findings represent a significant advancement in ASD genetics, as we discovered many genes involved in the repeat expansions that had not been previously identified using conventional genomic analyses (Table 2). Beyond implications for ASD, we have revealed far more extensive variability among such sequences than previously recognized in the human genome, with 7.7% of the repeats interrogated having more than one motif detected. This suggests that some genes may be prone to expansions with different repeat motifs.

Coupling repeat identification with an outlier detection method, we were able to identify 2,483 repeat-containing regions that, when expanded, occur in genes involved in biological functions and pathways, such as those involved in nervous system, cardiovascular system and muscle. For example, there was a correlation between the repeat expansions and shaping cognitive and behavioral phenotypes in ASD. For example, DMPK, in which rare SNVs and CNVs were found in individuals with ASD, had not been conclusively linked to ASD previously, because the majority of ASD-relevant alterations were not detected until the expanded repeats were analyzed in our study. Notably, many of the ASD-relevant repeat expansions we discovered are in the non-coding regions of genes, and their mechanisms of gene regulation and aberrant splicing have been well-established (e.g., DMPK and FXN).

While allowing sensitive and accurate detection of the expanded repeat sequence, the method we developed here provides an estimated relative aggregated length of the repeat tracts.

### Data Availability

Access to the MSSNG and SSC genome sequencing data can be obtained by completing data access agreements (https://research.mss.ng and https://www.sfari.org/resource/sfari-base, respectively). The 1000G genome sequencing data are publicly available via Amazon Web Services (s3://1000genomes/1000G_2504_high_coverage/data).

### Example 2

The methods described in Example were employed to identify outlier tandem repeat sequences in an epilepsy population.

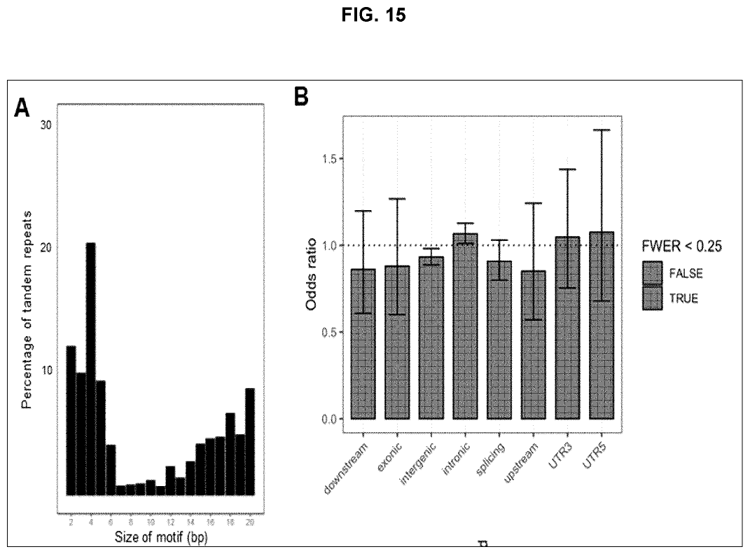

As shown in FIG. 15A, the distribution of motif sizes in tandem repeats was comparable to that identified in ASD. Various outlier tandem repeat sequences were identified, those of significance being present in intergenic or intronic gene regions (FIG. 15B).

### Example 3

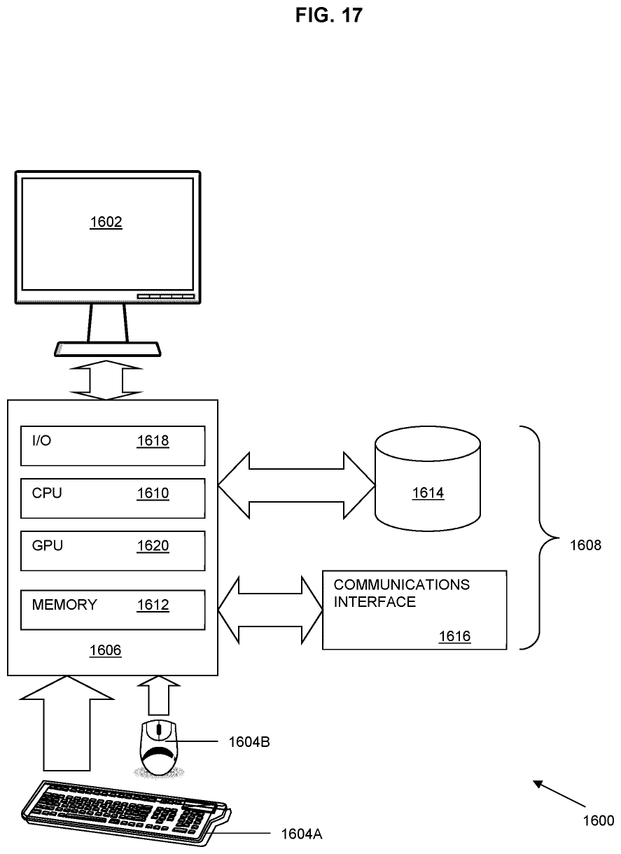

A method of operating a gene sequencer is described for the identification of outlier tandem repeat sequences in a population of interest. Reference is made to FIG. 17, which pictorially illustrates a method, indicated generally by reference 1500, for operating a genetic sequencer, indicated generally by reference 1502. The genetic sequencer 1502 may be, for example and without limitation, one of the various models offered by Illumina, Inc. having an address at 5200 Illumina Way, San Diego, Calif. 92122.

Initially, a physical nucleic acid sample 1504 is obtained, for example from individuals in a population of interest. The individuals may be human individuals, or non-human individuals. The physical nucleic acid sample 1504 may, for example and without limitation, come from a blood sample, tissue or other sample as described herein. Next, the nucleic acid samples 1504 are prepared 1506 for the genetic sequencer 1502 to obtain prepared nucleic acid samples 1508. The precise mode of preparation 1506 will depend on the type and model of the genetic sequencer 1502 and will be within the capability of one of ordinary skill in the art, now informed by the present disclosure. The prepared nucleic acid samples 1508 are then input 1510 into the genetic sequencer 1502, which sequences the prepared nucleic acid samples 1508 to obtain nucleic acid sequence information 1512 for the population of interest from the prepared nucleic acid samples 1508.

The method 1500 analyzes the nucleic acid sequence information using analysis logic 1514 to detect in the nucleic acid sequence information 1512 the presence of one or more outlier sample tandem repeat sequences 1516, as described further below.

In one embodiment, the analysis of the nucleic acid sequence information 1512 is carried out solely by the genetic sequencer 1502, for example one or more onboard processors of the genetic sequencer 1502 may execute the analysis logic 1514, which may reside in storage media of the genetic sequencer 1502. In another embodiment, the analysis of the nucleic acid sequence information 1512 is carried out by the genetic sequencer 1502 in conjunction with at least one external computer system 1518 communicatively coupled 1520 to the genetic sequencer 1508. For example the analysis logic 1514 may be executed in part by one or more onboard processors of the genetic sequencer 1502 and in part by one or more processors of the external computer system(s) 1518. In still another embodiment, the analysis of the nucleic acid sequence 1512 is carried out solely by the external computer system(s) 1518, which receives the nucleic acid sequence information 1512 obtained by the genetic sequencer 1502.

The analysis logic 1514 may have a number of implementations.

In one such implementation, the analysis logic 1514 detects sample tandem repeat sequences comprising a repeated motif sequence in the nucleic acid sequence information. These sample tandem repeat sequences have a length that permits a tandem repeat calling algorithm (e.g. within the analysis logic 1514) to detect and/or genotype the tandem repeat sequences. The analysis logic 1514 may simulate the length distribution of population of interest tandem repeat sequences in a population of interest to a normal distribution if no sample tandem repeat sequences are detected initially. The analysis logic 1514 then detects one or more outlier sample tandem repeat sequences 1516, and may also determine the disease with which the outlier tandem repeat sequence is associated based on the location of tandem repeat sequence within the genome, including the gene in which it is present. An outlier sample tandem repeat sequence(s) has a length that is greater than that in 90% of the population of interest tandem repeat sequences detected in the population of interest and occur at a frequency of less than about 1% of control population tandem repeat sequences detected in a control population.

In another implementation, the analysis logic 1514 detects in the nucleic acid sequence of an individual the presence of one or more outlier sample tandem repeat sequences 1516 determined to be prominent for a disease and, responsive to detecting the presence of the outlier sample tandem repeat sequence(s), determines that the individual has the disease. Responsive to determining that the individual has the disease, the method 1500 may emit a signal 1522 that the individual has the disease. The signal 1522 may be one or more of an audible signal, a visual signal (e.g. a flashing light or on-screen display), and an electronic communication (e.g. an e-mail message, text message, SMS message, iMessage, or the like). These are merely non-limiting illustrative examples of a signal 1522.

The foregoing are merely illustrative examples of analysis logic 1514 and are not intended to be limiting; the analysis logic 1514 may incorporate other aspects of the present disclosure as well.

As can be seen from the above description, the method 1500 described above represents significantly more than merely using categories to organize, store and transmit information and organizing information through mathematical correlations. The method 1500 in fact an improvement to the technology of genetic sequencing and genetic analysis, as it provides for detection of outlier sample tandem repeat sequences within a nucleic acid sequence, which may facilitate disease detection. Moreover, the method 1500 is applied by using a particular machine, namely a genetic sequencer. As such, the method 1500 is confined to genetic sequencing applications.

Aspects of the present disclosure may be embodied within a system, a method, a computer program product or any combination thereof. The computer program product may include a computer readable storage medium or media having computer readable program instructions thereon for causing a processor to carry out aspects of the present technology. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present technology may be assembler instructions, instruction-set-architecture (ISA) instructions, machine instructions, machine dependent instructions, microcode, firmware instructions, state-setting data, or either source code or object code written in any combination of one or more programming languages, including an object oriented programming language or a conventional procedural programming language. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to implement aspects of the present technology.

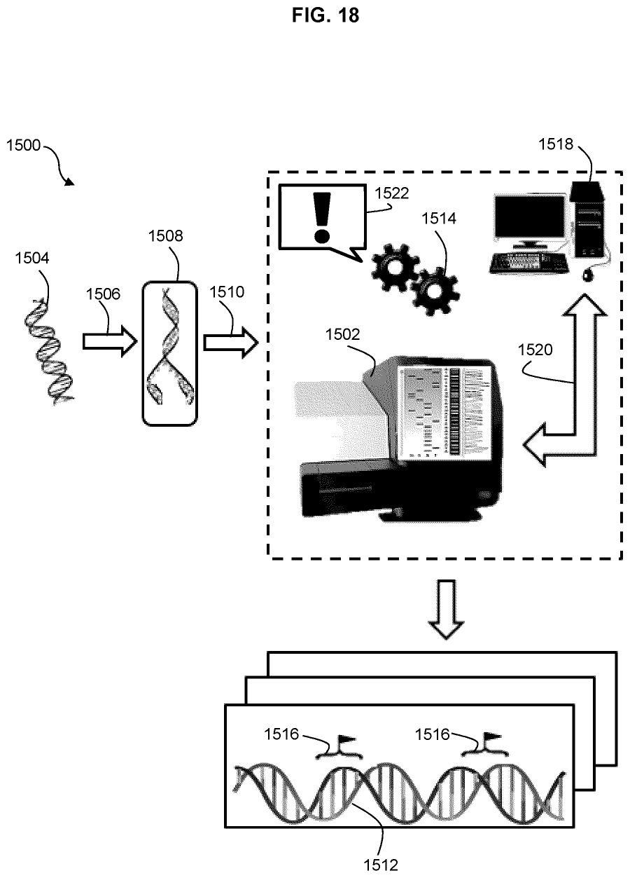

An illustrative computer system in respect of which the technology herein described may be implemented is presented as a block diagram in FIG. 18. The illustrative computer system is denoted generally by reference numeral 1600 and includes a display 1602, input devices in the form of keyboard 1604A and pointing device 1604B, computer 1606 and external devices 1608. While pointing device 1604B is depicted as a mouse, it will be appreciated that other types of pointing device, or a touch screen, may also be used. For example, the computer system 1600 may represent an illustrative embodiment of the external computer system(s) 1518 shown in FIG. 17.

The computer 1606 may contain one or more processors or microprocessors, such as a central processing unit (CPU) 1610. The CPU 1610 performs arithmetic calculations and control functions to execute software stored in an internal memory 1612, preferably random access memory (RAM) and/or read only memory (ROM), and possibly additional memory 1614. The additional memory 1614 may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces such as those found in video game devices, removable memory chips such as EPROM or PROM, emerging storage media, such as holographic storage, or similar storage media as known in the art. This additional memory 1614 may be physically internal to the computer 1606, or external as shown in FIG. 18, or both.

The computer system 1600 may also include other similar means for allowing computer programs or other instructions to be loaded. Such means can include, for example, a communications interface 1616 which allows software and data to be transferred between the computer system 1600 and external systems and networks. Examples of communications interface 1616 can include a modem, a network interface such as an Ethernet card, a wireless communication interface, or a serial or parallel communications port. Software and data transferred via communications interface 1616 are in the form of signals which can be electronic, acoustic, electromagnetic, optical or other signals capable of being received by communications interface 1616. Multiple interfaces, of course, can be provided on a single computer system 1600.

Input and output to and from the computer 1606 is administered by the input/output (I/O) interface 1618. This I/O interface 1618 administers control of the display 1602, keyboard 1604A, external devices 1608 and other such components of the computer system 1600. The computer 1606 also includes a graphical processing unit (GPU) 1620. The latter may also be used for computational purposes as an adjunct to, or instead of, the (CPU) 1610, for mathematical calculations.

The various components of the computer system 1600 are coupled to one another either directly or by coupling to suitable buses. The term “computer system”, “data processing system” and related terms, as used herein, is not limited to any particular type of computer system and encompasses servers, desktop computers, laptop computers, networked mobile wireless telecommunication computing devices such as smartphones, tablet computers, as well as other types of computer systems. Thus, computer readable program code for implementing aspects of the technology described herein may be contained or stored in the memory 1612 of the computer 1606, or on a computer usable or computer readable medium external to or the computer 1606, or on any combination thereof.

Finally, the terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed.

The description has been presented for purposes of illustration and description, but is not intended to be exhaustive or limited to the form disclosed. Many modifications and variations will be apparent to those of ordinary skill in the art without departing from the scope of the claims. The embodiment was chosen and described in order to best explain the principles of the technology and the practical application, and to enable others of ordinary skill in the art to understand the technology for various embodiments with various modifications as are suited to the particular use contemplated.

One or more currently preferred embodiments have been described by way of example. It will be apparent to persons skilled in the art that a number of variations and modifications can be made without departing from the scope of the claims. In construing the claims, it is to be understood that the use of a computer (including an onboard computer of a genetic sequencer) to implement certain of the embodiments described herein is essential.

### REFERENCES

1 Yuen, R. K. et al. Whole genome sequencing resource identifies 18 new candidate genes for autism spectrum disorder. Nat Neurosci 20, 602-611, doi:10.1038/nn.4524 (2017).
2 Fischbach, G. D. & Lord, C. The Simons Simplex Collection: a resource for identification of autism genetic risk factors. Neuron 68, 192-195, doi:10.1016/j.neuron.2010.10.006 (2010).
3 1000 Genomes Project Consortium. A global reference for human genetic variation. Nature 526, 68-74, doi:10.1038/nature15393 (2015).
4 Li, H. & Durbin, R. Fast and accurate short read alignment with Burrows-Wheeler transform. Bioinformatics 25, 1754-1760, doi:10.1093/bioinformatics/btp324 (2009).
5 Levy, S. et al. The diploid genome sequence of an individual human. PLoS Biol 5, e254, doi:10.1371/journal.pbio.0050254 (2007).
6 Alexander, D. H., Novembre, J. & Lange, K. Fast model-based estimation of ancestry in unrelated individuals. Genome Res 19, 1655-1664, doi:10.1101/gr.094052.109 (2009).
7 Benson, G. Tandem repeats finder: a program to analyze DNA sequences. Nucleic Acids Res 27, 573-580, doi:10.1093/nar/27.2.573 (1999).
8 Koren, S. et al. Canu: scalable and accurate long-read assembly via adaptive k-mer weighting and repeat separation. Genome Res 27, 722-736, doi:10.1101/gr.215087.116 (2017).
9 Ester, M., Kriegel, H., Sander, J. & Xu, X. A density-based algorithm for discovering clusters a density-based algorithm for discovering clusters in large spatial databases with noise. Proceedings of the Second International Conference on Knowledge Discovery and Data Mining 226-231 (AAAI Press, Portland, Oreg., 1996).
10 Wang, K., Li, M. & Hakonarson, H. ANNOVAR: functional annotation of genetic variants from high-throughput sequencing data. Nucleic Acids Res 38, e164, doi:10.1093/nar/gkq603 (2010).

1. A method of detecting one or more outlier tandem repeat sequence associated with a disease in a population of interest comprising the following steps:
i) detecting tandem repeat sequences comprising a repeated motif sequence in nucleic acid samples from individuals within the population of interest, wherein the tandem repeat sequences have a length that permits a tandem repeat calling algorithm to detect and/or genotype the tandem repeat sequences;
ii) simulating the length distribution of the tandem repeat sequences in the population of interest to a normal distribution if no tandem repeat sequences are detected in step i); and
iii) detecting one or more outlier tandem repeat sequences, wherein an outlier tandem repeat sequence has a length that is greater than that in 90% of the tandem repeat sequences detected in the population interest and occur at a frequency of less than 1% of the tandem repeat sequences detected in a control population.

2. The method of claim 1, wherein the repeated motif sequence comprises 1-25 base pairs and occur directly adjacent to one another.

3. The method of claim 2, wherein the repeated motif sequence comprises 2 to 20 base pairs.

4. The method of claim 1, wherein the tandem repeat sequence is greater than 150 base pairs in length.

5. The method of claim 1, wherein the outlier tandem repeat sequence comprises a GC content of at least about 10% in comparison to the tandem repeat sequences detected.

6. The method of claim 1, wherein the outlier tandem repeat sequence is located within an intronic region of a gene, and/or located within about 10,000 base pairs to a transcriptional start site (TSS) or a splice junction.

7. The method of claim 1, wherein the disease is selected from a developmental disorder in the brain, blood, heart or kidney, neurological disease, cardiomyopathy, cancer and diabetes.

8. The method of claim 1, wherein the disease is a neuropsychiatric disorder.

9. The method of claim 1, wherein the disease is Autism Spectrum Disorder (ASD).

10. A method of diagnosing or determining risk of a target disease in an individual comprising the steps of:
i) conducting a method as defined in claim 1 to identify outlier tandem repeat sequences associated with disease;
ii) detecting the presence of one or more outlier tandem repeat sequences in nucleic acid in the individual, and diagnosing the individual with the target disease, or at risk of the target disease.

11. A method of diagnosing or determining risk of a target disease in an individual comprising the steps:
i) detecting in a nucleic acid sample from the individual the presence of a target outlier tandem repeat sequence determined to be prominent for the target disease using a nucleic acid probe that specifically binds thereto, or binds to a sequence adjacent thereto;
ii) determining that the individual has the disease or is at risk of the disease when the target outlier tandem repeat sequence is present in the nucleic acid sample; and
iii) optionally, treating the individual using an appropriate therapy for the disease.

12. A method of operating a genetic sequencer, the method comprising:
sequencing prepared nucleic acid samples from individuals within a population of interest using the genetic sequencer to obtain nucleic acid sequence information comprising nucleic acid sequences for each prepared nucleic acid sample; and
analyzing the nucleic acid sequence information by:
i) detecting tandem repeat sequences comprising a repeated motif sequence in the nucleic acid sequence information, wherein the tandem repeat sequences have a length that permits a tandem repeat calling algorithm to detect and/or genotype the tandem repeat sequences;
ii) simulating the length distribution of population tandem repeat sequences in the population of interest to a normal distribution if no tandem repeat sequences are detected in step i); and
iii) detecting one or more outlier tandem repeat sequences, wherein an outlier sample tandem repeat sequence has a length that is greater than that in 90% of the tandem repeat sequences detected in the population of interest and occur at a frequency of less than 1% of the tandem repeat sequences detected in a control population.

13. The method of claim 12, wherein analyzing the nucleic acid sequence information is carried out solely by the genetic sequencer.

14. The method of claim 12, wherein analyzing the nucleic acid sequence information is carried out solely by a computer that receives the nucleic acid sequence information obtained by the genetic sequencer.

15. The method of claim 12, wherein analyzing the nucleic acid sequence information is carried out by the genetic sequencer in conjunction with at least one external computer system coupled to the genetic sequencer.

16. A method of operating a genetic sequencer, the method comprising:
obtaining a physical nucleic acid sample for an individual;
preparing the nucleic acid sample for the genetic sequencer to obtain a prepared nucleic acid sample;
inputting the prepared nucleic acid sample into the genetic sequencer;
sequencing the prepared nucleic acid sample by the genetic sequencer to obtain a nucleic acid sequence for the prepared nucleic acid sample; and
analyzing the nucleic acid sequence by:
i) detecting in the nucleic acid sequence the presence of one or more outlier sample tandem repeat sequences determined to be unique for a disease;
ii) responsive to detecting in the nucleic acid sequence the presence of the one or more outlier sample tandem repeat sequences is present, determining that the individual has the disease; and responsive to determining that the individual has the disease, emitting a signal that the individual has the disease.

17. The method of claim 16, wherein the signal is at least one of an audible signal, a visual signal, and an electronic communication.

18. The method of claim 16, wherein the individual is a human individual.

19. The method of claim 16, wherein analyzing the nucleic acid sequence is carried out solely by the genetic sequencer.

20. The method of claim 16, wherein analyzing the nucleic acid sequence is carried out solely by a computer that receives the nucleic acid sequence obtained by the genetic sequencer.

21. The method of claim 16, wherein analyzing the nucleic acid sequence is carried out by the genetic sequencer in conjunction with at least one external computer system coupled to the genetic sequencer.
